(19) United States
(12) Patent Application Publication
(10) Pub. No.: US 2018/0002705 A1
(43) Pub. Date: Jan. 4, 2018

(54) FUSION PROTEIN AND TRANSGENIC PLANT EXPRESSING SAID PROTEIN

(71) Applicant: Università degli Studi di Roma "La Sapienza", Roma (IT)

(72) Inventors: Felice Cervone, Roma (IT); Giulia De Lorenzo, Roma (IT); Simone Ferrari, Roma (IT); Manuel Benedetti, Roma (IT); Daniela Pontiggia, Roma (IT)

(21) Appl. No.: 15/538,646

(22) PCT Filed: Dec. 22, 2015
(86) PCT No.: PCT/EP2015/081017

§ 371 Date: Jun. 21, 2017

(30) Foreign Application Priority Data
Dec. 23, 2014 (IT) RM2014A000748

(51) Int. Cl. C12N 15/62; A01N 25/10; C07K 14/435; C07K 14/37; C07K 14/195; C12N 15/82; C07K 14/415

(52) U.S. Cl. CPC C12N 15/62; C12N 15/8286; C12N 15/8282; C12N 15/8281; A01N 25/10; C07K 14/415; C07K 14/37; C07K 14/195; C07K 14/43563

(57) ABSTRACT

The present invention concerns a nucleic acid molecule capable of expressing, in at least one plant tissue, a chimeric protein comprising a polygalacturonase (PG) of fungal, bacterial or insect origin and a plant polygalacturonase inhibitor protein (PGIP) plant capable of inhibiting said PG. The present invention also relates to transgenic plants that express said chimeric protein.

### RELATED CASES

This application is the national stage entry under 35 U.S.C. §371 of International Patent Application No. PCT/EP2015/081017, filed on Dec. 22, 2015, which claims the benefit of Italian Patent Application No. RM2014A000748, filed on Dec. 23, 2014, the entirety of each of which is incorporated herein by reference.

### TECHNICAL FIELD

The present invention concerns a nucleic acid molecule capable of expressing, in at least one plant tissue, a chimeric protein comprising a polygalacturonase (PG) of fungal, bacterial or insect origin and a plant polygalacturonase inhibitor protein (PGIP) capable of inhibiting said PG. The present invention also relates to transgenic plants that express said chimeric protein.

### PRIOR ART

Plant immunity is mediated not only by pathogen-derived molecules, called microbe-associated molecular patterns (MAMPs) (1), but also by endogenous molecules referred to as damage-associated molecular patterns (DAMPs), which are released by the host cell during a microbial infection (2-4).

Oligogalacturonides (OGs), oligomers of α-1,4-galacturonic acid released by the plant cell wall following a partial hydrolysis of homogalacturonan (HG), which is the main constituent of pectin, are the best characterised of all DAMPs (3).

Most information on OGs has been obtained from in vitro experiments using exogenous treatments; direct evidence of their accumulation and their function in plants has to date been lacking.

In the last 30 years evidence has been provided of the fact that OGs applied from the outside are capable of activating defence responses in plant tissues (2,3,5). It has been speculated that the enzymatic degradation of homogalacturonan, which takes place during microbial infections, leads to a OG-mediated defence response of the plant against pathogens. It has been demonstrated in vitro that the interaction between polygalacturonase (PG) and the polygalacturonase inhibitor protein (PGIP), located in the cell wall, can favour the accumulation of OGs with eliciting activity (4,6). The use of PGIP has since been tested in transgenic plants (7-9), where the resistance provided by the inhibitor against a fungus is dependent on the specificity of recognition of the inhibitor, which enables it to inhibit only some of the various existing fungal polygalacturonases (10,11).

U.S. Pat. No. 5,569,830 relates to nucleotide sequences encoding plant polygalacturonase inhibitor proteins (PGIP) which inhibit the activity of fungal polygalacturonases. Transgenic plants expressing a heterologous PGIP show an increased resistance to the fungi that normally infect plants.

Patent application EP0577252 relates to a method of creating a transgenic tomato containing a lowered level of the isoform 1 of polygalacturonase. The DNA sequence encoding at least a portion of the polygalacturonase beta-subunit which is sufficient to hybridise effectively to the mRNA for the polygalacturonase beta-subunit in vivo is used to create a construct in which the cDNA is positioned in such a way as to produce the antisense version of the polygalacturonase beta-subunit message.

Moreover, the hypothesis that PGIP favors the production of OGs in vivo and that these molecules in turn act as defence signals during an infection has never been proven (Benedetti et al., Proceedings of the National Academy of Sciences, Vol. 112, no. 17, 2015).

Further, plants simultaneously expressing the PGII of Aspergillus niger and the PGIP2 of Phaseolus vulgaris, which is able to inhibit the PGII of A. niger, obtained by crossing two transgenic plants separately expressing either PG or PGIP, do not allow the production of OG in vivo (Ferrari S, Galletti R, Pontiggia D, Manfredini C, Lionetti V, Bellincampi D, Cervone F, De Lorenzo G: Transgenic expression of a fungal endo-polygalacturonase increases plant resistance to pathogens and reduces auxin sensitivity. Plant Physiol 2008, 146:669-681).

The basic concept is founded on evidence deriving from the use of OGs obtained in vitro from commercial pectins originating from tissues of different species of plants. OGs with a degree of polymerisation (DP) of between 10 and 15 activate a wide range of defence responses, such as the accumulation of phytoalexins (12-14), glucanase and chitinase (15, 16), as well as the expression of genes correlated with defence (17,18) and the production of reactive oxygen species (19, 20). In the model plant Arabidopsis, OGs are perceived by the wall-associated receptor kinase WAK1 (21, 22), activate gene expression independent of the signalling pathway which involves ethylene, salicylic acid and jasmonic acid (23) and activate the phosphorylation of the MAP kinases AtMPK3 and AtMPK6 (24). OGs can moreover induce a strong “oxidative burst” mediated by NADPH oxidase AtRbohD, which is also partly involved in the consequent accumulation of callose in the cell wall (20). Similarly, in the cells of mammals, DAMP signals deriving from the degradation of hyaluronan in the extracellular matrix activate inflammatory responses through the kinase receptors TLR2 and TLR4, which are also required for the perception of MAMPs (25). Therefore, MAMPs and DAMPs, notwithstanding their origin and distinctive characteristics, are functionally similar both in plants and animals (26).

### DETAILED DESCRIPTION OF THE INVENTION

The authors have demonstrated that Arabidopsis plants expressing a chimeric protein obtained from the fusion between a fungal PG and a PGIP accumulate OGs in plant tissues and hence activate the plant defense responses. Furthermore, plants expressing this fusion protein (called OG-machine, initials OGM), under the control of a pathogen-inducible promoter, have an increased resistance against pathogenic microorganisms such as fungi and bacteria. The present data demonstrate that it is possible to engineer the release of DAMPs so as to be able to induce plant immunity in vivo. OGMs and DAMPs are thus powerful tools capable of providing protection to the plant against pathogens.

It is therefore an object of the present invention a nucleic acid molecule coding for a chimeric protein comprising:

a) an amino acid sequence with a polygalacturonase inhibitor (PGIP) activity of plant origin,

b) an amino acid sequence with a polygalacturonase (PG) activity as in a) of fungal, bacterial or insect origin.

Preferably, said chimeric protein comprises the sequence a) at the N-terminal portion and the sequence b) at the C-terminal portion.

In a preferred embodiment of the invention, the nucleic acid molecule codes for a chimeric protein wherein the amino acid sequence with the PGIP activity comprises a sequence selected from the group consisting of:

the sequence of PGIP2 of Phaseolus vulgari (Pv PGIP2) comprising the sequence SEQ ID NO:4, or a functional fragment of the same responsible for the polygalacturonase inhibitor activity, a isoform thereof or a functional equivalent, variant, mutant, derivative, synthetic or recombinant functional analogue thereof;

the sequence of PGIP1 of Phaseolus vulgari (Pv PGIP1) comprising the sequence SEQ ID NO:23, or a functional fragment of the same responsible for the polygalacturonase inhibitor activity or a isoform thereof or a functional equivalent, variant, mutant, derivative, synthetic or recombinant functional analogue thereof;

the sequence of PGIP3 of Phaseolus vulgari (Pv PGIP3) comprising the sequence SEQ ID NO:25, or a functional fragment of the same responsible for the polygalacturonase inhibitor activity or a isoform thereof or a functional equivalent, variant, mutant, derivative, synthetic or recombinant functional analogue thereof;

the sequence of the PGIP of Malus domestica comprising the sequence SEQ ID NO: 26, or a functional fragment of the same responsible for the polygalacturonase inhibitor activity or a isoform thereof or functional equivalent, variant, mutant, derivative, synthetic or recombinant functional analogue thereof;

the sequence of PGIP1 of Vitis vinifera comprising the sequence SEQ ID NO: 28, or a functional fragment of the same responsible for the polygalacturonase inhibitor activity or a isoform thereof or functional equivalent, variant, mutant, derivative, synthetic or recombinant functional analogue thereof; and

the sequence of PGIP1 or PGIP2 of Arabidopsis thaliana comprising respectively the sequence SEQ ID NO:30 or 31, or a functional fragment of the same responsible for the polygalacturonase inhibitor activity or a isoform thereof or a functional equivalent, variant, mutant, derivative, synthetic or recombinant functional analogue thereof.

In a more preferred embodiment of the invention, the amino acid sequence with the PGIP activity comprises the sequence of PGIP2 of Phaseolus vulgaris (Pv PGIP2) comprising or having essentially the sequence SEQ ID NO:4, or a functional fragment of the same responsible for the polygalacturonase inhibitor activity or isoforms thereof.

In a preferred embodiment of the invention, the nucleic acid molecule codes for a chimeric protein wherein the amino acid sequence with the PG activity comprises a sequence selected from the group consisting of:

the sequence of PG of Fusarium phyllophilum (FpPG) comprising the sequence SEQ ID NO:2 or SEQ ID NO:22, or a functional fragment of the same responsible for the polygalacturonase activity or a isoform thereof or functional equivalent, variant, mutant, derivative, synthetic or recombinant functional analogue thereof;

the sequence of PG2 of Aspergillus niger comprising the sequence SEQ ID NO:24 or a functional fragment of the same responsible for the polygalacturonase activity or a isoform thereof or a functional equivalent, variant, mutant, derivative, synthetic or recombinant functional analogue thereof;

the sequence of the PG of Colletotrichum lupini comprising the sequence SEQ ID NO:27 or a functional fragment of the same responsible for the polygalacturonase activity or a isoform thereof or a functional equivalent, variant, mutant, derivative, synthetic or recombinant functional analogue thereof; and

the sequence of BcPG2 of Botrytis cinerea comprising the sequence SEQ ID NO:29 or a functional fragment of the same responsible for the polygalacturonase activity or a isoform thereof or functional equivalent, variant, mutant, derivative, synthetic or recombinant functional analogue thereof.

In a more preferred embodiment of the invention, the amino acid sequence with the PG activity comprises the sequence of PG of Fusarium phyllophilum (FpPG) comprising or having essentially the sequence SEQ ID NO:2, or a functional fragment of the same responsible for the polygalacturonase activity or isoforms thereof.

In a preferred embodiment of the invention, the nucleic acid molecule codes for a chimeric protein wherein the amino acid sequence with the PGIP activity comprises the sequence of PGIP2 of Phaseolus vulgari (Pv PGIP2) comprising the sequence SEQ ID NO:4, or a functional fragment of the same responsible for the polygalacturonase inhibitor activity or a isoform thereof

and wherein the amino acid sequence with the PG activity comprises the sequence of PG of Fusarium phyllophilum (FpPG) comprising the sequence SEQ ID NO:2, or a functional fragment of the same responsible for the polygalacturonase activity or a isoform thereof.

In a more preferred embodiment of the invention, the nucleic acid molecule codes for a chimeric protein, comprising:

a) the SEQ ID NO:4 and

b) the SEQ ID NO:2.

Preferably, the functional fragment of the sequence SEQ ID NO:4, has essentially the sequence aa. 30-aa. 342 of SEQ ID NO:4.

In an alternative preferred embodiment, the amino acid sequence with the PGIP activity comprises the sequence of PVPGIP1 of Phaseolus vulgaris comprising the sequence SEQ ID NO:23 or the sequence of PGIP2 of Phaseolus vulgari (Pv PGIP2) comprising the sequence SEQ ID NO:4, or a functional fragment of the same responsible for the polygalacturonase inhibitor activity or a isoform thereof and wherein the amino acid sequence with the PG activity comprises the sequence of PG2 of Aspergillus niger comprising the sequence SEQ ID NO:24 or a functional fragment of the same responsible for the polygalacturonase activity or a isoform thereof.

In an alternative preferred embodiment, the amino acid sequence with the PGIP activity comprises the sequence of the PGIP of Malus domestica comprising the sequence SEQ ID NO:26, or a functional fragment of the same responsible for the polygalacturonase inhibitor activity or a isoform thereof and wherein the amino acid sequence with the PG activity comprises the sequence of the PG of Colletotrichum lupini comprising the sequence SEQ ID NO:27 or a functional fragment of the same responsible for the polygalacturonase activity or a isoform thereof.

In a further preferred embodiment of the invention, the amino acid sequence with the PGIP activity comprises the sequence of PGIP1 of Vitis vinifera comprising the sequence SEQ ID NO:28, or a functional fragment of the same responsible for the polygalacturonase inhibitor activity or a isoform thereof and wherein the amino acid sequence with the PG activity comprises the sequence of BcPG2 of Botrytis cinerea comprising the sequence SEQ ID NO: 29 or a functional fragment of the same responsible for the polygalacturonase activity or a isoform thereof.

In another preferred embodiment of the invention, the amino acid sequence with the PGIP activity comprises the sequence of PGIP1 or PGIP2 of Arabidopsis thaliana comprising the sequence SEQ ID NO:30 or 31 respectively, or a functional fragment of the same responsible for the polygalacturonase inhibitor activity or a isoform thereof and wherein the amino acid sequence with the PG activity comprises the sequence of BcPG2 of Botrytis cinerea comprising the sequence SEQ ID NO: 29 or a functional fragment of the same responsible for the polygalacturonase activity or a isoform thereof.

Preferably, the nucleic acid molecule according to the invention comprises a region coding for a linker, preferably comprised between the sequence coding for the amino acid sequence with PGIP activity and the sequence coding for the amino acid sequence with PG activity.

More preferably, said linker is of sequence Ala, Ala, Ala.

Preferably, the nucleic acid molecule according to the invention comprises a region coding for a signal peptide, preferably derived from bean or yeast.

In a preferred embodiment, the nucleic acid molecule to the invention comprises the nucleotide sequence having essentially the SEQ ID NO: 5, SEQ ID NO: 7 or SEQ ID NO: 9.

Preferably, the nucleic acid molecule as above defined further comprises a promoter which is active in plants. Said promoter is preferably pathogen inducible, more preferably it is the promoter which regulates the expression of PR-1 gene of Arabidopsis (PPR-1) (PPR-1 [Genbank: Accession number: CP002685.1, from 6242431 bp to 6243722 bp]). Preferably, said promoter comprises or has essentially the sequence nt.1-1291 of the SEQ ID NO: 9. Other preferred promoters are e.g.: promoter which regulates the expression of VSR gene of Arabidopsis (PVSR) (PVSR [Genbank: Accession number: CP002684.1, from 10996136 bp to 10997274 bp]), promoter which regulates the expression of PBS2/RAR1 gene of Arabidopsis (PPBS2/RAR1) (PPBS2/RAR1 [Genbank: Accession number: CP002688.1, from 21003143 bp to 21004278 bp]).

Another object of the invention is an expression vector comprising the nucleic acid molecule as above defined. In said expression vector, the nucleic acid molecule is preferably under the control of a promoter which is active in plants. Said promoter is preferably pathogen inducible, more preferably it is the promoter which regulates the expression of PR-1 gene of Arabidopsis (PPR-1) (PPR-1 [Genbank: Accession number: CP002685.1, from 6242431 bp to 6243722 bp]). Preferably, said promoter comprises or has essentially the sequence nt.1-1291 of the SEQ ID NO: 9.

Other preferred promoters are e.g.: promoter which regulates the expression of VSR gene of Arabidopsis (PVSR) (PVSR [Genbank: Accession number: CP002684.1, from 10996136 bp to 10997274 bp]), promoter which regulates the expression of PBS2/RAR1 gene of Arabidopsis (PPBS2/RAR1) (PPBS2/RAR1 [Genbank: Accession number: CP002688.1, from 21003143 bp to 21004278 bp]).

Another object of the invention is the use of the vector as described above or of the nucleic acid molecule as described above for producing transgenic plants or transformed plant tissues or transformed plant cells.

A further object of the invention is a transgenic plant obtainable through the use as described above, or parts thereof.

Another object of the invention is a transgenic plant, or parts thereof, comprising the nucleic acid molecule as described above and/or expressing the chimeric protein or functional fragments thereof according to the invention.

Another object of the invention are the seeds of the transgenic plant of the invention.

Another object of the invention is a chimeric protein, or a functional fragment of the same, as described above.

A further object of the invention is a chimeric protein, or a functional fragment of the same, coded by the nucleic acid molecule as described above.

Preferably, said chimeric protein comprises the amino acid sequence having essentially the SEQ ID NO: 6 or the SEQ ID NO: 8 or functional fragment or equivalent, variant, mutant, derivative, synthetic or recombinant functional analogue thereof.

Another object of the invention is a genetically engineered host cell comprising the nucleic acid molecule as described above or the vector as described above and/or expressing the chimeric protein as described above.

The functional fragment of the amino acid sequence with the PGIP activity can comprise, for example, at least 20, 25, 35, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330 or 340 aa of the SEQ ID NO: 4, 23, 25, 26, 28, 30 or 31.

| The amino acid sequence with the PG activity comprising the sequence | |
|---|---|
| of PG of Fusarium phyllophilum (FpPG) can alternatively have the | |
| sequence of NCBI Uniprot Accession Number: Q07181.1: | |
| (SEQ ID NO: 22) | |
| ORIGIN | |
| 1 | mvrnivsrlc sqlfalpsss lqerdpcsvt eysglatavs scknivlngf qvptgkqldi |
| 61 | sslqudstvt fkgtttfatt adndfnpivi sgsnititga sghvidgngq aywdgkgsns |
| 121 | nsnqkpdhfi vvqkttgnsk itnlniqnwp vhcfditgss qltisglild nragdkpnak |
| 181 | sgslpaahnt dgfdisssdh vtldnnhvyn qddcvavtsg trtiwsnmyc sgghglsigs |
| 241 | vggksdnvvd gvcgflssqw nsqngcriks nsgatgtinn vtyqniaitn istygvdvqq |
| 301 | dyinggptgk ptngvkisni kfikvtgtva ssaqdwfilc gdgscsgftf sgnaitgggk |
| 361 | tssenyptnt cps. |
| // | |

The functional fragment of the amino acid sequence with the PG activity can comprise, for example, at least 20, 25, 35, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330 or 340 aa of the SEQ ID NO: 2, 22, 24, 27 or 29.

Preferably, the nucleic acid molecule according to the invention comprises the nucleotide sequence having essentially the SEQ ID NO:3 and/or the SEQ ID NO:1.

The above described linker is preferably 2-10 amino acid long, more preferably 3-8 amino acid long, even more preferably it is 3 amino acid long.

Preferably, said linker comprises hydrophobic aminoacids. More preferably, said linker is of the sequence Ala, Ala, Ala.

The linker as above defined may be selected by the skilled in the art according to its properties. In particular, said linker should be of a proper length which allows to avoid the proteolytic cleavage of the chimeric protein. Said linker allows the equimolar production of the amino acid sequence with PGIP activity and of the amino acid sequence with PG activity. The linker also allows the intermolecular interaction between the PGIP and the PG moieties, while not permitting intramolecular enzyme-inhibitor interactions.

The chimeric protein according to the invention, is preferably expressed under the control of a promoter that is non constitutive and which is preferably active only during the infection.

Said promoter is preferably the promoter which regulates the expression of the gene PR-1 of Arabidopsis (PPR-1 [Genbank: Accession number: CP002685.1, from 6242431 bp to 6243722 bp]). Preferably, said promoter comprises or has essentially the sequence nt.1-1291 of the SEQ ID NO: 9.

The nucleic acid of the invention allows to obtain the expression of both PGIP and PG simultaneously and in equimolar amounts.

The transgenic plants according to the invention thus accumulate equimolar levels of PG and PGIP.

As used here, the term “nucleic acid” refers to RNA or DNA, preferably DNA. Said DNA can be double-stranded or single-stranded. The term also includes the complementary strand of the specified sequences. The nucleic acid molecule of the invention can also include additional coding sequences, such as a leader sequence or a pro-protein sequence, and/or additional non-coding sequences, such as UTR sequences.

The term “nucleic acid” can also refer to a “vector”, such as, for example, an expression vector. The term “expression vector” comprises, for example, a plasmid, a viral particle, a phage, etc. Such vectors can include bacterial plasmids, phagic DNA, baculovirus, yeast plasmids, vectors derived from combinations of plasmids and phage DNA, and viral DNA, such as vaccinia, adenovirus, fowl pox virus and pseudorabies virus. A large number of suitable vectors are known to the person skilled in the art and are commercially available.

The nucleic acid molecule, preferably the DNA sequence, in the vector is operatively linked to an appropriate expression control sequence (promoter) for direct mRNA synthesis. As examples representative of such promoters, one may mention a prokaryote or eukaryote promoter such as a CMV immediate-early promoter, HSV thymidine kinases, early and late SV40 and retrovirus LTR. The promoter is preferably the above defined promoter of PR-1. The expression vector also contains a ribosomal binding site for starting the translation and a transcription vector. The vector can also include sequences appropriate for the expression of amplification.

Furthermore, the vectors preferably contain one or more marker genes that can be selected to provide a phenotypic trait for the selection of the transformed host cell, e.g. dihydrofolate reductase or neomycin resistance for the culture of eukaryotic cells, or tetracycline or ampicillin resistance in E. coli.

As used here, the term “genetically engineered host cell” refers to host cells that have been transduced, transformed or transfected with the nucleic acid molecule or with the vector previously described.

As representative examples of appropriate host cells, one may mention bacterial cells such as E. coli, Streptomyces and Salmonella typhimurium cells, fungal cells such as yeast cells, insect cells such as Sf9 cells, animal cells such as CHO or COS cells, plant cells etc. The selection of an appropriate host is considered in the field of application by the person skilled in the art. Preferably, said host cell is a plant cell.

The nucleic acid molecule or vector previously described can be introduced into the host cell using methods that are well known to a person skilled in the art, e.g. transfection with calcium phosphate, DEAE-dextran mediated transfection, biolistic particle bombardment, transformation mediated by Agrobacterium tumefaciens or electroporation.

The nucleic acid molecule of the invention can comprise a region coding for a linker capable of favouring intra- or intermolecular interaction in the chimeric protein.

In the context of the present invention the terms “protein”, “amino acid sequence with PGIP activity” or “amino acid sequence with PG activity” include:

i. the whole protein (for example pvPGIP2 [Uniprot Accession Number: P58822.1], or the SEQ ID NO:4, or FpPG [Uniprot Accession Number: Q07181.1] or the SEQ ID NO:2, or the other PGIPs and PGs above described), isoform, allelic variants and proteins coded by an orthologous gene of the same (for example, proteins coded by an orthologous gene of Pv PGIP2 or of SEQ ID NO:4, or proteins coded by an orthologous gene of FpPG or of SEQ ID NO:2 or of the other PGIPs and PGs above described);

ii. any functional fragment of the protein with an inhibiting activity of PG, in the case of PGIP protein fragments, or polygalacturonase activity, in the case of PG protein fragments;

iii. any equivalent, variant, mutant, functional derivative, synthetic or recombinant functional analogue, with PG-inhibiting activity in the case of equivalents, variants, mutants, functional derivatives, synthetic or recombinant functional analogues of the PGIP protein or of the amino acid sequence with PGIP activity (or of SEQ ID NO: 4 or of the sequences of the other PGIPs above described) or with polygalacturonase activity in the case of equivalents, variants, mutants, functional derivatives and synthetic or recombinant functional analogues of the PG protein or of the amino acid sequence with PG activity (or of SEQ ID NO: 2 or of the sequences of the other PGs above described).

In the context of the present invention, the term “functional fragment” of the chimeric protein as described above refers to fragments which, once expressed in a plant, plant tissue or plant cells are capable of inducing the release of oligogalacturonides (OGs). Said fragment may be long 100, 200, 250, 300, 350, 400, 450, 500, 550, 600 amino acids.

The term “chimeric protein” also includes its functional equivalent, variant, mutant, derivative, synthetic or recombinant functional analogue.

The terms “mutant” or “derivative” or “variant”, as used in the context of the present invention can be intended as the substitution, deletion and/or addition of a single amino acid in the protein sequence. Preferably, the mutation of the protein sequence in the present invention is a substitution. The substitution can take place with a genetically coded amino acid or a non-genetically coded amino acid. Examples of non-genetically coded amino acids are homocysteine, hydroxyproline, omithin, hydroxylysine, citrulline, carnitine, etc.

As used here, the term “equivalent” means a peptide having at least one of the activities of the protein PGIP or PG or the same activity of the chimeric protein of the invention.

“Analogue” will be understood to mean a protein that exhibits some modifications relative to the proteins PG or PGIP. These modifications can be a deletion, a truncation, an extension, a chimeric fusion, and/or a mutation. Among the analogue proteins, those showing more than 80% of identity are preferred.

“Derivative” refers to any protein, possibly mutated, truncated, and/or extended, which was chemically modified or contains unusual amino acids.

As used here, the term “derivatives” refers also to proteins having a percentage of identity of at least 75% with the sequences disclosed in the present invention, e.g. with SEQ ID NO:2, SEQ ID NO:4, SEQ ID NO: 6 or SEQ ID NO:8, preferably at least 85%, for example at least 90%, and more preferably at least 95%.

The chimeric protein of the invention, if required, can be modified in vitro and/or in vivo, for example by glycosylation, myristoylation, amidation, carboxylation or phosphorylation, and can be obtained, for example, by means of synthetic or recombinant techniques known in the art.

As used here, the term “orthologues” refers to genes in different species relative to the gene coding for the proteins PvPGIP, or of SEQ ID NO:4, or FpPG, or of SEQ ID NO:2 or relative to the gene for the proteins as above defined. As examples of such orthologues, one may mention the proteins corresponding to PGIP in Arabidopsis thaliana, Nicotiana tabacum, Glycine max, Gossypium arboreum, Brassica napus, Vitis vinifera and Beta vulgaris or PG in Botrytis cinerea, Aspergillus niger, Colletotricum lupini, Fusarium oxysporum, Erwinia carotovora, Lygus ruguhpennis and Adelphocoris lineolatus.

In further preferred embodiments, the nucleic acid molecule of the invention codes for a protein comprising the sequences of:

| the isoform PVPGIP1 of Phaseolus vulgaris (NCBI Uniprot Accession Number | |
|---|---|
| P35334.1: | |
| ORIGIN | |
| 1 | mtqfnlpvtm ssslsillvi lvslrtalse lcnpqdkqal lqikkdlgnp ttlsswlptt |
| 61 | dccnrtwlgv lcdtdtqtyr vnnldlsghn lpkpypipss lanlpylnfl yigginnlvg |
| 121 | pippaiaklt alhylyitht nvsgaipdfl sqiktlvtld fsynalsgtl ppsisslpnl |
| 181 | ggitfdgnri sgaipdsygs fsklftamti srnrltgkip ptfanlnlaf vdlsrnmleg |
| 241 | dasvlfgsdk ntkkihlakn slafdlgkvg lsknlngldl rnnriygtlp qgltqlkflq |
| 301 | slnvsfnnlc geipqggnlk rfdvssyann kclcgsplps ct |
| // | |
| (SEQ ID NO: 23)) | |
| and | |
| the isoform PG2 of Aspergillus niger (Uniprot Accession Number P26214.1: | |
| ORIGIN | |
| 1 | mhsfasllay glvagatfas aspieardsc tfttaaaaka gkakcstitl nnievpagtt |
| 61 | ldltgltsgt kvifegtttf gyeewagpli smsgehitvt gasghlincd garwwdgkgt |
| 121 | sgkkkpkffy ahgldsssit glnikntplm afsvqandit ftdvtinnad gdtqgghntd |
| 181 | afdvgnsvgv niikpwvhnq ddclavnsge niwftggtci gghglsigsv gdrsnnvvkn |
| 241 | vtiehstvsn senavrikti sgatgsvsei tysnivmsgi sdygvviqqd yedgkptgkp |
| 301 | tngvtiqdvk lesvtgsvds gateiyllcg sgscsdwtwd dvkvtggkks tacknfpsva |
| 361 | sc |
| // | |
| (SEQ ID NO: 24)) (10); | |
| the PGIP of Malus domestica (NCBI Uniprot Accession Number P93270.1): | |
| ORIGIN | |
| 1 | melkfsifls ltllfssvlk palsdlcnpd dkkvllqikk afgdpyvlts wksdtdccdw |
| 61 | ycvtcdsttn rinsltifag qvsgqipalv gdlpyletle fhkqpnitgp iqpaiaklkg |
| 121 | lkflrlswtn lsgsvpdfls qlknitfldl sfnnitgaip sslsqlpnln alhldrnklt |
| 181 | ghipkslgqf ignvpdlyls hnqlsgnipt sfaqmdftsi dlsrnklegd asvifglnkt |
| 241 | tqivdlsrnl lefnlskvef ptsltsldin hnkiygsipv eftqlnfqfl nvsynrlcgq |
| 301 | ipvggklqsf deysyfhnrc lcgaplpsck |
| // | |
| (SEQ ID NO: 26)) | |
| and | |
| the PG of Colletotrichum lupini var setosum (NCBI Uniprot Accession | |
| Number A1E266.1): | |
| ORIGIN | |
| 1 | mvssllalga laataiaapl darasctftd aaaaikgkas ctsiilngiv vpagttldmt |
| 61 | glksgttvtf qgkttfgyke wegplisfsg tniningasg hsidcqgsrw wdskgsnggk |
| 121 | tkpkffyahs lkssnikgln vintpvqafs insattlgvy dviidnsagd sagghntdaf |
| 181 | dvgsstgvyi sganvknqdd clainsgtni tftggtcsgg hglsigsvgg rsdntvktvt |
| 241 | isnskivnsd ngvriktvsg atgsysgvty sgitlsniak ygivieqdye ngsptgtptn |
| 301 | gvpitgltls kitgsvassg tnvyilcasg acsnwkwsgv svtggkkstk csnipsgsga |
| 361 | ac |

//
(SEQ ID NO: 27))

(Oelofse D, Dubery I A, Meyer R, Arendse M S, Gazendam I, Berger D K: Apple polygalacturonase inhibiting protein1 expressed in transgenic tobacco inhibits polygalacturonases from fungal pathogens of apple and the anthracnose pathogen of lupins. Phytochem 2006, 67:255-263.);

| the isoform PGIP1 of Vitis vinifera (NCBI Uniprot Accession Number | |
|---|---|
| A7PW81.1): | |
| ORIGIN | |
| 1 | metsklflls sslllvllat rpcpslserc npkdkkvllq ikkaldtpyi laswnpntdc |
| 61 | cgwycvecdl tthrinslti fsgqlsgqip davgdlpfle tlifrklsnl tgqippaiak |
| 121 | lkhlkmvrls wtnlfgpvpa ffselknity ldlsfnnlsg pipgslsllp nlgalhidrn |
| 181 | hltgpipdsf gkfagstpgl hlshnqlsgk ipysfrgfdp nvmdlsrnkl egdlsiffna |
| 241 | nkstqivdfs rnlfqfdlsr vefpksltsl dlshnkiags 1pemmtsldl qfinvsynr1 |
| 301 | cgkipvggkl qsfdydsyfh nrcicgaplq sck |
| // | |
| (SEQ ID NO: 28)) | |
| and | |
| the isoform BcPG2 of Botrytis cinerea (NCBI Uniprot Accession Number | |
| A4VB48.1): | |
| ORIGIN | |
| 1 | mvhitslisf lastalvsaa pgsapadldr ragctfstaa taiaskttcs tiildsvvvp |
| 61 | agttldltgl ktgtkvifqg tatfgysewe gplisisgqd ivvtgasgnk idgggarwwd |
| 121 | glgsnvsagk gkvkpkffsa hkltgsssit glnflnapvq cisiggsvgl slininidns |
| 181 | agdagnlghn tdafdinlsq nifisgaivk nqddcvavns gtnitftggn csgghglsig |
| 241 | svggrsgtga ndvkdvrfls stvqkstngv rvktvsdtkg svtgvtfqdi tligitgvgi |
| 301 | dvqqdyqngs ptgtptngvp itgltmnnvh gnviggqnty ilcancsgwt wnkvavtggt |
| 361 | vkkacagvpt gasc |

//
(SEQ ID NO: 29))

(Joubert D A, Kars I, Wagemakers L, Bergmann C, Kemp G, Vivier M A, van Kan J A L: A polygalacturonase-inhibiting protein from grapevine reduces the symptoms of the endopolygalacturonase BcPG2 from Botrytis cinerea in Nicotiana benthamiana leaves without any evidence for in vitro interaction. Mol Plant-Microbe Interact 2007, 20:392-402);

| The isoforms PGIP1 or PGIP2 of Arabidopsis thaliana (NCBI Uniprot | |
|---|---|
| Accession Number Q9M5J9.1 and Q9M5J8.2, respectively]): | |
| ORIGIN | |
| 1 | mdktatlcll flftflttcl skdlcnqndk ntllkikksl nnpyhlaswd pqtdccswyc |
| 61 | lecgdatvnh rvtaltifsg qisgqipaev gdlpyletiv frklsnitgt iqptiaklkn |
| 121 | lrmlrlswtn ltgpipdfis qlknleflel sfndlsgsip sslstlpkil alelsrnklt |
| 181 | gsipesfgsf pgtvpdlrls hnqlsgpipk slgnidfnri dlsrnklqgd asmlfgsnkt |
| 241 | twsidlsrnm fqfdiskvdi pktlgildln hngitgnipv qwteaplqff nvsynklcgh |
| 301 | iptggklqtf dsysyfhnkc lcgapleick |
| // | |
| (SEQ ID NO: 30) | |
| ORIGIN | |
| 1 | mdktmtlfll lstlllttsl akdlchkddk ttllkikksl nnpyhlaswd pktdccswyc |
| 61 | lecgdatvnh rvtsliiqdg eisgqippev gdlpyltsli frkltnitgh iqptiaklkn |
| 121 | ltflrlswtn ltgpvpefls qlknleyidl sfndlsgsip sslsslrkle ylelsrnklt |
| 181 | gpipesfgtf sgkvpslfls hnqlsgtipk slgnpdfyri dlsrnklqgd asilfgakkt |
| 241 | twivdisrnm fqfdlskvkl aktlnnldmn hngitgsipa ewskayfqll nvsynrlcgr |
| 301 | ipkgeyiqrf dsysffhnkc lcgaplpsck |

//
(SEQ ID NO: 31))

and the isoform BcPG2 of Botrytis cinerea (NCBI Uniprot Accession Number A4VB48.1) (SEQ ID NO:29)) (Ferrari S, Galletti R, Denoux C, De Lorenzo G, Ausubel F M, Dewdney J: Resistance to Botrytis cinerea induced in Arabidopsis by elicitors is independent of salicylic acid, ethylene, or jasmonate signaling but requires PHYTOALEXIN DEFICIENT3. Plant Physiol 2007, 144:367-379)

or any other combinations of PG and PGIP known to the expert in the art.

| A further isoform of PGIP included in the present invention is, for | |
|---|---|
| example, PvPGIP3 (Uniprot Accession Number P58823.1: | |
| ORIGIN: | |
| 1 | mtqfnipvtm ssslsiilvi lvslrtalse lcnpqdkqal lqikkdlgnp ttlsswlptt |
| 61 | dccnrtwlgv lcdtdtqtyr vnnldlsghn lpkpypipss lanlpylnfl yigginnlvg |
| 121 | pippaiaklt qlhylyitht nvsgaipdfl sqiktlvtld fsynalsgtl ppsisslpnl |
| 181 | vgitfdgnri sgaipdsygs fsklftsmti srnrltgkip ptfanlnlaf vdlsrnmlqg |
| 241 | dasvlfgsdk ntqkihlakn sldfdlekvg lsknlngldl rnnriygtlp qgltqlkflh |
| 301 | slnvsfnnlc geipqggnlq rfdvsayann kclcgsplpa ct |

//
(SEQ ID NO: 25)).

The nucleic acid molecule according to the invention also comprises sequences having 70%, 80%, 90%, 95% or 100% identity sequence with the nucleotide sequence of SEQ ID NO: 5, SEQ ID NO: 7 or SEQ ID NO: 9.

The transgenic plants according to the invention are resistant to bacterial and/or fungal and/or insect infections, produced e.g. by Botrytis cinerea, Pectobacterium carotovorum and Pseudomonas syringae p.v. tabaci DC3000.

In the context of the present invention the “plant” may be e.g. a plant included in the genera Arabidopsis, as e.g. Arabidopsis thaliana, or Phaseolus, as e.g. Phaseolus vulgaris, or Nicotiana, as e.g. Nicotiana tabacum, or Glycine, as e.g. Glycine max, or Gossypium, as e.g. Gossypium arboreum, or Brassica, as e.g. Brassica napus, or Vitis, as e.g. Vitis vinifera, or Beta, as e.g. Beta vulgaris, or Triticum, as e.g. Triticum aestivum, or Solanum, as e.g. Solanum lycopersicum, Solanum tuberosum and Solanum melongena L., or Musa, as e.g. Musa acuminata and Musa balbisiana, or Fragaria, as e.g. Fragaria vesca, Fragaria viridis and Fragaria moschata, or Oryza, as e.g. Oryza sativa, or Hordeum, as e.g. Hordeum vulgare, or Olea, as e.g. Olea europaea.

In the context of the present invention fungal origin means that the protein is of e.g. a fungus included in the genera Fusarium, as e.g. Fusarium oxysporum, Fusarium phyllophiliu, Aspergillus, as e.g. Aspergillus niger, Botrytis, as e.g. Botrytis cinerea, Colletotrichum, as e.g. Colletotrichum lupine.

In the context of the present invention bacterial origin means that the protein is of e.g. a bacterium included in the genera Erwinia, as e.g. Erwinia carotovora.

In the context of the present invention bacterial origin means that the protein is of e.g. a insect included in the genera Lygus, as e.g. Lygus rugulipennis, and Adelphocoris, as e.g. Adelphocoris lineolatus.

SEQUENCES

NUCLEOTIDE SEQUENCE of Fusarium phyllophilum PG
(cDNA without the sequence coding for the
signal peptide and the proteolytic cleavage
signal [“GAT” nucleotides])
(SEQ ID NO: 1)
CCCTGCTCCGTGACTGAGTACTCTGGCCTCGCCACCGCTGTCTCATCCT
GCAAAAACATCGTGCTCAACGGTTTCCAAGTCCCGACAGGCAAGCAACT
CGACCTATCCAGCCTCCAGAATGACTCGACCGTTACCTTCAAGGGCACG
ACCACTTTTGCCACCACTGCTGATAACGACTTTAATCCTATCGTCATTA
GTGGAAGTAACATCACTATCACTGGTGCATCTGGCCATGTCATTGATGG
CAACGGTCAGGCGTACTGGGATGGCAAAGGTTCTAACAGCAATAGCAAC
CAAAAGCCCGATCACTTCATCGTTGTTCAGAAGACCACCGGCAACTCAA
AGATCACAAACCTAAATATCCAGAACTGGCCCGTTCACTGCTTCGACAT
TACAGGCAGCTCGCAATTGACCATCTCAGGGCTTATTCTTGATAACAGA
GCTGGCGACAAGCCTAACGCCAAGAGCGGTAGCTTGCCCGCTGCGCATA
ACACCGACGGTTTCGACATCTCGTCCAGTGACCACGTTACGCTGGATAA
CAATCATGTTTATAACCAAGATGATTGTGTTGCTGTTACTTCCGGTACA
AACATCGTCGTTTCTAACATGTATTGCTCCGGCGGCCATGGTCTTAGTA
TCGGATCTGTTGGTGGAAAGAGCGACAATGTCGTTGATGGTGTTCAGTT
CTTGAGCTCGCAGGTTGTGAACAGTCAGAATGGATGTCGCATCAAGTCC
AACTCTGGCGCAACTGGCACGATCAACAACGTCACCTACCAGAACATTG
CTCTCACCAACATCAGCACGTACGGTGTCGATGTTCAGCAGGACTATCT
CAACGGCGGCCCTACTGGAAAGCCGACCAACGGAGTCAAGATCAGCAAC
ATCAAGTTCATCAAGGTCACTGGCACTGTGGCTAGCTCTGCCCAGGATT
GGTTTATTCTGTGTGGTGATGGTAGCTGCTCTGGATTTACCTTCTCTGG
AAACGCTATTACTGGTGGTGGCAAGACTAGCAGCTGCAACTATCCTACC
AACACTTGCCCCAGCTAG
AMINO ACID Sequence of Fusarium phyllophilum PG
(without the amino acid sequence coding for the
signal peptide and amino acid D, proteolytic
cleavage signal) (SEQ ID NO: 2, corresponding to
region aa. 26-aa. 373 of the sequence with
Accession No. NCBI Q07181.1)
ORIGIN

| | |
|---|---|
| 1 | PCSVTEYSGL ATAVSSCKNI |
| 21 | VLNGFQVPTG KQLDLSSLQN |
| 41 | DSTVTFKGTT TFATTADNDF |
| 61 | NPIVISGSNI TITGASGHVI |
| 81 | DGNGQAYWDG KGSNSNSNQK |
| 101 | PDHFIVVQKT TGNSKITNLN |
| 121 | IQNWPVHCFD ITGSSQLTIS |
| 141 | GLILDNRAGD KPNAKSGSLP |
| 161 | AAHNTDGFDI SSSDHVTLDN |
| 181 | NHVYNQDDCV AVTSGTNIVV |
| 201 | SNMYCSGGHG LSIGSVGGKS |
| 221 | DNVVDGVQFL SSQVVNSQNG |
| 241 | CRIKSNSGAT GTINNVTYQN |
| 261 | IALTNISTYG VDVQQDYLNG |
| 281 | GPTGKPTNGV KISNIKFIKV |
| 301 | TGTVASSAQD WFILCGDGSC |
| 321 | SGFTFSGNAI TGGGKTSSCN |
| 341 | YPTNTCPS* |

NUCLEOTIDE SEQUENCE of Phaseolus vulgaris PGIP2
(SEQ ID NO: 3)
ATGACTCAATTCAATATCCCAGTAACCATGTCTTCAAGCTTAAGCATAA
TTTTGGTCATTCTTGTATCTTTGAGCACTGCACACTCAGAGCTATGCAA
CCCACAAGACAAGCAAGCCCTTCTCCAAATCAAGAAAGACCTTGGCAAC
CCAACCACTCTCTCCTCATGGCTTCCAACCACCGACTGTTGCAACAGAA
CCTGGCTAGGTGTTTTATGCGACACCGACACCCAAACATATCGCGTCAA
CAACCTCGACCTCTCCGGCCTTAACCTCCCAAAACCCTACCCTATCCCT
TCCTCCCTCGCCAACCTCCCCTACCTCAATTTTCTATACATTGGTGGCA
TCAATAACCTCGTCGGTCCAATCCCCCCCGCCATCGCTAAACTCACCCA
ACTCCACTATCTCTATATCACCCACACCAATGTCTCCGGCGCAATACCC
GATTTCTTGTCACAGATCAAAACCCTCGTCACCCTCGACTTCTCCTACA
ACGCCCTCTCCGGCACCCTACCTCCCTCCATCTCTTCTCTCCCCAACCT
CGTCGGAATCACATTCGACGGCAACCGAATCTCCGGCGCCATCCCCGAC
TCCTACGGCTCATTTTCGAAGCTGTTCACGTCGATGACCATCTCCCGCA
ACCGCCTCACCGGGAAGATTCCGCCGACGTTTGCGAATCTGAACCTGGC
GTTCGTTGACTTGTCTCGAAACATGCTGGAGGGTGACGCGTCGGTGTTG
TTCGGATCAGATAAGAACACGCAGAAGATACATCTGGCGAAGAACTCTC
TTGCCTTTGATTTGGGGAAAGTGGGGTTGTCAAAGAACTTGAACGGGTT
GGATCTGAGGAACAACCGTATCTATGGGACGCTACCGCAGGGACTGACG
CAGCTAAAGTTTCTGCACAGTTTAAATGTGAGCTTCAACAATCTGTGCG
GTGAGATTCCTCAAGGTGGGAACTTGCAAAGATTTGACGTTTCTGCTTA
TGCCAACAACAAGTGCTTGTGTGGTTCTCCTCTTCCTGCCTGCACT
AMINO ACID SEQUENCE of Phaseolus vulgaris PGIP2
(SEQ ID NO: 4, Accession No. NCBI UNIPROT
P58822.1)
ORIGIN

| | |
|---|---|
| 1 | MTQFNIPVTM SSSLSIILVI |
| 21 | LVSLSTARSE LCNPQDKQAL |
| 41 | LQIKKDLGNP TTLSSWLPTT |
| 61 | DCCNRTWLGV LCDTDTQTYR |
| 81 | VNNLDLSGLN LPKPYPIPSS |
| 101 | LANLPYLNFL YIGGINNLVG |
| 121 | PIPPAIAKLT QIHYLYITHT |
| 141 | NVSGAIPDFL SQIKTLVTLD |
| 161 | FSYNALSGTL PPSISSLPNL |
| 181 | VGITFDGNRI SGAIPDSYGS |
| 201 | FSKLFTSMTI SRNRLTGKIP |
| 221 | PTFANLNLAF VDLSRNMLEG |
| 241 | DASVLFGSDK NTQKIHLAKN |
| 261 | SLAFDLGKVG LSKNINGLDL |
| 281 | RNNRIYGTLP QGLTQLKFLH |
| 301 | SLNVSFNNLC GEIPQGGNLQ |
| 321 | RFDVSAYANN KCLCGSPLPA |
| 341 | CT* |

NUCLEOTIDE SEQUENCE OF THE OGM EXPRESSED
IN PLANTS
(SEQ ID NO: 5)
ATGACTCAATTCAATATCCCAGTAACCATGTCTTCAAGCTTAAGCATAA
TTTTGGTCATTCTTGTATCTTTGAGCACTGCACACTCAGAGCTATGCAA
CCCACAAGACAAGCAAGCCCTTCTCCAAATCAAGAAAGACCTTGGCAAC
CCAACCACTCTCTCCTCATGGCTTCCAACCACCGACTGTTGCAACAGAA
CCTGGCTAGGTGTTTTATGCGACACCGACACCCAAACATATCGCGTCAA
CAACCTCGACCTCTCCGGCCTTAACCTCCCAAAACCCTACCCTATCCCT
TCCTCCCTCGCCAACCTCCCCTACCTCAATTTTCTATACATTGGTGGCA
TCAATAACCTCGTCGGTCCAATCCCCCCCGCCATCGCTAAACTCACCCA
ACTCCACTATCTCTATATCACCCACACCAATGTCTCCGGCGCAATACCC
GATTTCTTGTCACAGATCAAAACCCTCGTCACCCTCGACTTCTCCTACA
ACGCCCTCTCCGGCACCCTACCTCCCTCCATCTCTTCTCTCCCCAACCT
CGTCGGAATCACATTCGACGGCAACCGAATCTCCGGCGCCATCCCCGAC
TCCTACGGCTCATTTTCGAAGCTGTTCACGTCGATGACCATCTCCCGCA
ACCGCCTCACCGGGAAGATTCCGCCGACGTTTGCGAATCTGAACCTGGC
GTTCGTTGACTTGTCTCGAAACATGCTGGAGGGTGACGCGTCGGTGTTG
TTCGGATCAGATAAGAACACGCAGAAGATACATCTGGCGAAGAACTCTC
TTGCCTTTGATTTGGGGAAAGTGGGGTTGTCAAAGAACTTGAACGGGTT
GGATCTGAGGAACAACCGTATCTATGGGACGCTACCGCAGGGACTGACG
CAGCTAAAGTTTCTGCACAGTTTAAATGTGAGCTTCAACAATCTGTGCG
GTGAGATTCCTCAAGGTGGGAACTTGCAAAGATTTGACGTTTCTGCTTA
TGCCAACAACAAGTGCTTGTGTGGTTCTCCTCTTCCTGCCTGCACT [symbol]
CCCTGCTCCGTGACTGAGTACTCTGGCCTCGCCACCGCTG
TCTCATCCTGCAAAAACATCGTGCTCAACGGTTTCCAAGTCCCGACAGG
CAAGCAACTCGACCTATCCAGCCTCCAGAATGACTCGACCGTTACCTTC
AAGGGCACGACCACTTTTGCCACCACTGCTGATAACGACTTTAATCCTA
TCGTCATTAGTGGAAGTAACATCACTATCACTGGTGCATCTGGCCATGT
CATTGATGGCAACGGTCAGGCGTACTGGGATGGCAAAGGTTCTAACAGC
AATAGCAACCAAAAGCCCGATCACTTCATCGTTGTTCAGAAGACCACCG
GCAACTCAAAGATCACAAACCTAAATATCCAGAACTGGCCCGTTCACTG
CTTCGACATTACAGGCAGCTCGCAATTGACCATCTCAGGGCTTATTCTT
GATAACAGAGCTGGCGACAAGCCTAACGCCAAGAGCGGTAGCTTGCCCG
CTGCGCATAACACCGACGGTTTCGACATCTCGTCCAGTGACCACGTTAC
GCTGGATAACAATCATGTTTATAACCAAGATGATTGTGTTGCTGTTACT
TCCGGTACAAACATCGTCGTTTCTAACATGTATTGCTCCGGCGGCCATG
GTCTTAGTATCGGATCTGTTGGTGGAAAGAGCGACAATGTCGTTGATGG
TGTTCAGTTCTTGAGCTCGCAGGTTGTGAACAGTCAGAATGGATGTCGC
ATCAAGTCCAACTCTGGCGCAACTGGCACGATCAACAACGTCACCTACC
AGAACATTGCTCTCACCAACATCAGCACGTACGGTGTCGATGTTCAGCA
GGACTATCTCAACGGCGGCCCTACTGGAAAGCCGACCAACGGAGTCAAG
ATCAGCAACATCAAGTTCATCAAGGTCACTGGCACTGTGGCTAGCTCTG
CCCAGGATTGGTTTATTCTGTGTGGTGATGGTAGCTGCTCTGGATTTAC
CTTCTCTGGAAACGCTATTACTGGTGGTGGCAAGACTAGCAGCTGCAAC
TATCCTACCAACACTTGCCCCAGCTAG
Legend:
underlined: SEQUENCE CODING FOR PGIP2
italics: SEQUENCE CODING FOR FpPG
bold: sequence coding for the spacer composed of
3 alanines
AMINO ACID SEQUENCE OF OGM EXPRESSED IN PLANTS
(inducible both by b-estradiol and under the
control of the promoter PR-1)
(SEQ ID NO: 6)
ORIGIN

| | |
|---|---|
| 1 | MTQFNIPVTM SSSLSIILVI |
| 21 | LVSLSTAHSE LCNPQDKQAL |
| 41 | LQIKKDLGNP TTLSSWLPTT |
| 61 | DCCNRTWLGV LCDTDTQTYR |
| 81 | VNNLDLSGLN LPKPYPIPSS |
| 101 | LANLPYLNFL YIGGINNLVG |
| 121 | PIPPAIAKLT QLHYLYITHT |
| 141 | NVSGAIPDFL SQIKTLVTLD |
| 161 | FSYNALSGTL PPSISSLPNL |
| 181 | VGITFDGNRI SGAIPDSYGS |
| 201 | FSKLFTSMTI SRNRLTGKIP |
| 221 | PTFANLNLAF VDLSRNMLEG |
| 241 | DASVLFGSDK NTQKIHLAKN |
| 261 | SLAFDLGKVG LSKNLNGLDL |
| 281 | RNNRIYGTLP QGLTQLKFLH |
| 301 | SLNVSFNNLC GEIPQGGNLQ |
| 321 | RFDVSAYANN KCLCGSPLPA |
| 341 | CTAAAPCSVT EYSGLATAVS |
| 361 | SCKNIVLNGF QVPTGKQLDL |
| 381 | SSLQNDSTVT FKGTTTFATT |
| 401 | ADNDFNPIVI SGSNITITGA |
| 421 | SGHVIDGNGQ AYWDGKGSNS |
| 441 | NSNQKPDHFI VVQKTTGNSK |
| 461 | ITNLNIQNWP VHCFDITGSS |
| 481 | QLTISGLILD NRAGDKPNAK |
| 501 | SGSLPAAHNT DGFDISSSDH |
| 521 | VTLDNNHVYN QDDCVAVTSG |
| 541 | TNIVVSNMYC SGGHGLSIGS |
| 561 | VGGKSDNVVD GVQFLSSQVV |
| 581 | NSQNGCRIKS NSGATGTINN |
| 601 | VTYQNIALTN ISTYGVDVQQ |
| 621 | DYLNGGPTGK PTNGVKISNI |
| 641 | KFIKVTGTVA SSAQDWFILC |
| 661 | GDGSCSGFTF SGNAITGGGK |
| 681 | TSSCNYPTNT CPS* |

Legend:
underlined: AMINO ACID SEQUENCE OF PGIP2
italics: AMINO ACID SEQUENCE OF FpPG
bold: AMINO ACID SEQUENCE FOR THE SPACER COMPOSED
OF 3 ALANINES
NUCLEOTIDE SEQUENCE OF OGM EXPRESSED IN PICHIA
(SEQ ID NO: 7)
GAGCTATGCAACCCACAAGACAAGCAAGCCCTTCTCCAAATCAAGAAAG
ACCTTGGCAACCCAACCACTCTCTCCTCATGGCTTCCAACCACCGACTG
TTGCAACAGAACCTGGCTAGGTGTTTTATGCGACACCGACACCCAAACA
TATCGCGTCAACAACCTCGACCTCTCCGGCCTTAACCTCCCAAAACCCT
ACCCTATCCCTTCCTCCCTCGCCAACCTCCCCTACCTCAATTTTCTATA
CATTGGTGGCATCAATAACCTCGTCGGTCCAATCCCCCCCGCCATCGCT
AAACTCACCCAACTCCACTATCTCTATATCACCCACACCAATGTCTCCG
GCGCAATACCCGATTTCTTGTCACAGATCAAAACCCTCGTCACCCTCGA
CTTCTCCTACAACGCCCTCTCCGGCACCCTACCTCCCTCCATCTCTTCT
CTCCCCAACCTCGTCGGAATCACATTCGACGGCAACCGAATCTCCGGCG
CCATCCCCGACTCCTACGGCTCATTTTCGAAGCTGTTCACGTCGATGAC
CATCTCCCGCAACCGCCTCACCGGGAAGATTCCGCCGACGTTTGCGAAT
CTGAACCTGGCGTTCGTTGACTTGTCTCGAAACATGCTGGAGGGTGACG
CGTCGGTGTTGTTCGGATCAGATAAGAACACGCAGAAGATACATCTGGC
GAAGAACTCTCTTGCCTTTGATTTGGGGAAAGTGGGGTTGTCAAAGAAC
TTGAACGGGTTGGATCTGAGGAACAACCGTATCTATGGGACGCTACCGC
AGGGACTGACGCAGCTAAAGTTTCTGCACAGTTTAAATGTGAGCTTCAA
CAATCTGTGCGGTGAGATTCCTCAAGGTGGGAACTTGCAAAGATTTGAC
GTTTCTGCTTATGCCAACAACAAGTGCTTGTGTGGTTCTCCTCTTCCTG
CCTGCACT [symbol] CCCTGCTCCGTGACTGAGTACTCTGGCCTCGC
CACCGCTGTCTCATCCTGCAAAAACATCGTGCTCAACGGTTTCCAAGTC
CCGACAGGCAAGCAACTCGACCTATCCAGCCTCCAGAATGACTCGACCG
TTACCTTCAAGGGCACGACCACTTTTGCCACCACTGCTGATAACGACTT
TAATCCTATCGTCATTAGTGGAAGTAACATCACTATCACTGGTGCATCT
GGCCATGTCATTGATGGCAACGGTCAGGCGTACTGGGATGGCAAAGGTT
CTAACAGCAATAGCAACCAAAAGCCCGATCACTTCATCGTTGTTCAGAA
GACCACCGGCAACTCAAAGATCACAAACCTAAATATCCAGAACTGGCCC
GTTCACTGCTTCGACATTACAGGCAGCTCGCAATTGACCATCTCAGGGC
TTATTCTTGATAACAGAGCTGGCGACAAGCCTAACGCCAAGAGCGGTAG
CTTGCCCGCTGCGCATAACACCGACGGTTTCGACATCTCGTCCAGTGAC
CACGTTACGCTGGATAACAATCATGTTTATAACCAAGATGATTGTGTTG
CTGTTACTTCCGGTACAAACATCGTCGTTTCTAACATGTATTGCTCCGG
CGGCCATGGTCTTAGTATCGGATCTGTTGGTGGAAAGAGCGACAATGTC
GTTGATGGTGTTCAGTTCTTGAGCTCGCAGGTTGTGAACAGTCAGAATG
GATGTCGCATCAAGTCCAACTCTGGCGCAACTGGCACGATCAACAACGT
CACCTACCAGAACATTGCTCTCACCAACATCAGCACGTACGGTGTCGAT
GTTCAGCAGGACTATCTCAACGGCGGCCCTACTGGAAAGCCGACCAACG
GAGTCAAGATCAGCAACATCAAGTTCATCAAGGTCACTGGCACTGTGGC
TAGCTCTGCCCAGGATTGGTTTATTCTGTGTGGTGATGGTAGCTGCTCT
GGATTTACCTTCTCTGGAAACGCTATTACTGGTGGTGGCAAGACTAGCA
GCTGCAACTATCCTACCAACACTTGCCCCAGCTAG
Legend:
underlined: SEQUENCE CODING FOR PGIP2
(corresponding to the region nt. 88-1026 of
SEQ ID NO: 3)
italics: SEQUENCE CODING FOR FpPG
bold: sequence coding for the spacer composed of
3 alanines
AMINO ACID SEQUENCE OF OGM EXPRESSED IN PICHIA
(SEQ ID NO: 8)
ORIGIN

| | |
|---|---|
| 1 | ELCNPQDKQA LLQIKKDLGN |
| 21 | PTTLSSWLPT TDCCNRTWLG |
| 41 | VLCDTDTQTY RVNNLDLSGL |
| 61 | NLPKPYPIPS SLANLPYLNF |
| 81 | LYIGGINNLV GPIPPAIAKL |
| 101 | TQLHYLYITH TNVSGAIPDF |
| 121 | LSQIKTLVTL DFSYNALSGT |
| 141 | LPPSISSLPN LVGITFDGNR |
| 161 | ISGAIPDSYG SFSKLFTSMT |
| 181 | ISRNRLTGKI PPTFANLNLA |
| 201 | FVDLSRNMLE GDASVLFGSD |
| 221 | KNTQKIHLAK NSLAFDLGKV |
| 241 | GLSKNLNGLD LRNNRIYGTL |
| 261 | PQGLTQLKFL HSLNVSFNNL |
| 281 | CGEIPQGGNL QRFDVSAYAN |
| 301 | NKCLCGSPLP ACTAAAPCSV |
| 321 | TEYSGLATAV SSCKNIVLNG |
| 341 | FQVPTGKQLD LSSLQNDSTV |
| 361 | TFKGTTTFAT TADNDFNPIV |
| 381 | ISGSNITITG ASGHVIDGNG |
| 401 | QAYWDGKGSN SNSNQKPDHF |
| 421 | IVVQKTTGNS KITNLNIQNW |
| 441 | PVHCFDITGS SQLTISGLIL |
| 461 | DNRAGDKPNA KSGSLPAAHN |
| 481 | TDGFDISSSD HVTLDNNHVY |
| 501 | NQDDCVAVTS GTNIVVSNMY |
| 521 | CSGGHGLSIG SVGGKSDNVV |
| 541 | DGVQFLSSQV VNSQNGCRIK |
| 561 | SNSGATGTIN NVTYQNIALT |
| 581 | NISTYGVDVQ QDYLNGGPTG |
| 601 | KPTNGVKISN IKFIKVTGTV |
| 621 | ASSAQDWFIL CGDGSCSGFT |
| 641 | FSGNAITGGG KTSSCNYPTN |
| 661 | TCPS* |

Legend:
underlined: PROTEIN SEQUENCE OF PGIP2
(corresponding to the region aa. 30-342 of the
SEQ ID NO: 4)
italics: PROTEIN SEQUENCE OF FpPG
bold: protein sequence coding of the spacer
composed of 3 alanines
NUCLEOTIDE SEQUENCE OF OGM FUSED TO PROMOTER PR-1
for the expression of the chimeric protein after
a pathogen attack
(SEQ ID NO 9)
AAGCTTGTTTTAACTTATAAAATGATTCTCCCTCCATATAAAAAAGTTT
GATTTTATAGAATGTTTATACCGATTAAAAAAATAATAATGCTTAGTTA
TAAATTACTATTTATTCATGCTAAACTATTTCTCGTAACTATTAACCAA
TAGTAATTCATCAAATTTTAAAATTCTCAATTAATTGATTCTTGAAATT
CATAACCTTTTAATATTGATTGATAAAAATATACATAAACTCAATCTTT
TTAATACAAAAAAACTTTAAAAAATCAATTTTTCTGATTCGGAGGGAGT
ATATGTTATTGCTTAGAATCACAGATTCATATCAGGATTGGAAAATTTT
AAAGCCAGTGCATATCAGTAGTCAAAATTGGTAAATGATATACGAAGGC
GGTACAAAATTAGGTATACTGAAGATAGAAGAACACAAAAGTAGATCGG
TCACCTAGAGTTTTTCAATTTAAACTGCGTATTAGTGTTTGGAAAAAAA
AAACAAAGTGTATACAATGTCAATCGGTGATCTTTTTTTTTTTTTTTTT
TTTTTTTTTTCTTTTTGGATAAATCTCAATGGGTGATCTATTGACTGTT
TCTCTACGTCACTATTTTACTTACGTCATAGATGTGGCGGCATATATTC
TTCAGGACTTTTCAGCCATAGGCAAGAGTGATAGAGATACTCATATGCA
TGAAACACTAAGAAACAAATAATTCTTGACTTTTTTTCTTTTATTTGAA
AATTGACTGTAGATATAAACTTTTATTTTTTCTGACTGTAAATATAATC
TTAATTGCCAAACTGTCCGATACGATTTTTCTGTATTATTTACAGGAAG
ATATCTTCAAAACATTTTGAATGAAGTAATATATGAAATTCAAATTTGA
AATAGAAGACTTAAATTAGAATCATGAAGAAAAAAAAAACACAAAACAA
CTGAATGACATGAAACAACTATATACAATGTTTCTTAATAAACTTCATT
TAGGGTATACTTACATATATACTAAAAAAATATATCAACAATGGCAAAG
CTACCGATACGAAACAATATTAGGAAAAATGTGTGTAAGGACAAGATTG
ACAAAAAAATAGTTACGAAAACAACTTCTATTCATTTGGACAATTGCAA
TGAATATTACTAAAATACTCACACATGGACCATGTATTTACAAAAACGT
GAGATCTATAGTTAACAAAAAAAAAAAGAAAAAAATAGTTTTCAAATCT
CTATATAAGCGATGTTTACGAACCCCAAAATCATAACACAACAATAACC
ATTATCAACTTAGAAAAATGACTCAATTCAATATCCCAGTAACCATGTC
TTCAAGCTTAAGCATAATTTTGGTCATTCTTGTATCTTTGAGCACTGCA
CACTCAGAGCTATGCAACCCACAAGACAAGCAAGCCCTTCTCCAAATCA
AGAAAGACCTTGGCAACCCAACCACTCTCTCCTCATGGCTTCCAACCAC
CGACTGTTGCAACAGAACCTGGCTAGGTGTTTTATGCGACACCGACACC
CAAACATATCGCGTCAACAACCTCGACCTCTCCGGCCTTAACCTCCCAA
AACCCTACCCTATCCCTTCCTCCCTCGCCAACCTCCCCTACCTCAATTT
TCTATACATTGGTGGCATCAATAACCTCGTCGGTCCAATCCCCCCCGCC
ATCGCTAAACTCACCCAACTCCACTATCTCTATATCACCCACACCAATG
TCTCCGGCGCAATACCCGATTTCTTGTCACAGATCAAAACCCTCGTCAC
CCTCGACTTCTCCTACAACGCCCTCTCCGGCACCCTACCTCCCTCCATC
TCTTCTCTCCCCAACCTCGTCGGAATCACATTCGACGGCAACCGAATCT
CCGGCGCCATCCCCGACTCCTACGGCTCATTTTCGAAGCTGTTCACGTC
GATGACCATCTCCCGCAACCGCCTCACCGGGAAGATTCCGCCGACGTTT
GCGAATCTGAACCTGGCGTTCGTTGACTTGTCTCGAAACATGCTGGAGG
GTGACGCGTCGGTGTTGTTCGGATCAGATAAGAACACGCAGAAGATACA
TCTGGCGAAGAACTCTCTTGCCTTTGATTTGGGGAAAGTGGGGTTGTCA
AAGAACTTGAACGGGTTGGATCTGAGGAACAACCGTATCTATGGGACGC
TACCGCAGGGACTGACGCAGCTAAAGTTTCTGCACAGTTTAAATGTGAG
CTTCAACAATCTGTGCGGTGAGATTCCTCAAGGTGGGAACTTGCAAAGA
TTTGACGTTTCTGCTTATGCCAACAACAAGTGCTTGTGTGGTTCTCCTC
TTCCTGCCTGCACT [symbol] [symbol] CCCTGCTCCGTGACTGAGTACTCTG
GCCTCGCCACCGCTGTCTCATCCTGCAAAAACATCGTGCTCAACGGTTT
CCAAGTCCCGACAGGCAAGCAACTCGACCTATCCAGCCTCCAGAATGAC
TCGACCGTTACCTTCAAGGGCACGACCACTTTTGCCACCACTGCTGATA
ACGACTTTAATCCTATCGTCATTAGTGGAAGTAACATCACTATCACTGG
TGCATCTGGCCATGTCATTGATGGCAACGGTCAGGCGTACTGGGATGGC
AAAGGTTCTAACAGCAATAGCAACCAAAAGCCCGATCACTTCATCGTTG
TTCAGAAGACCACCGGCAACTCAAAGATCACAAACCTAAATATCCAGAA
CTGGCCCGTTCACTGCTTCGACATTACAGGCAGCTCGCAATTGACCATC
TCAGGGCTTATTCTTGATAACAGAGCTGGCGACAAGCCTAACGCCAAGA
GCGGTAGCTTGCCCGCTGCGCATAACACCGACGGTTTCGACATCTCGTC
CAGTGACCACGTTACGCTGGATAACAATCATGTTTATAACCAAGATGAT
TGTGTTGCTGTTACTTCCGGTACAAACATCGTCGTTTCTAACATGTATT
GCTCCGGCGGCCATGGTCTTAGTATCGGATCTGTTGGTGGAAAGAGCGA
CAATGTCGTTGATGGTGTTCAGTTCTTGAGCTCGCAGGTTGTGAACAGT
CAGAATGGATGTCGCATCAAGTCCAACTCTGGCGCAACTGGCACGATCA
ACAACGTCACCTACCAGAACATTGCTCTCACCAACATCAGCACGTACGG
TGTCGATGTTCAGCAGGACTATCTCAACGGCGGCCCTACTGGAAAGCCG
ACCAACGGAGTCAAGATCAGCAACATCAAGTTCATCAAGGTCACTGGCA
CTGTGGCTAGCTCTGCCCAGGATTGGTTTATTCTGTGTGGTGATGGTAG
CTGCTCTGGATTTACCTTCTCTGGAAACGCTATTACTGGTGGTGGCAAG
ACTAGCAGCTGCAACTATCCTACCAACACTTGCCCCAGCTAG
Legend:
underlined: SEQUENCE OF THE PROMOTER PR-1
italics: SEQUENCE CODING FOR THE OGM
bold: SEQUENCE CODING FOR THE LINKER OF 3
ALANINES

The present invention will be illustrated with non-limiting examples in reference to the following figures.

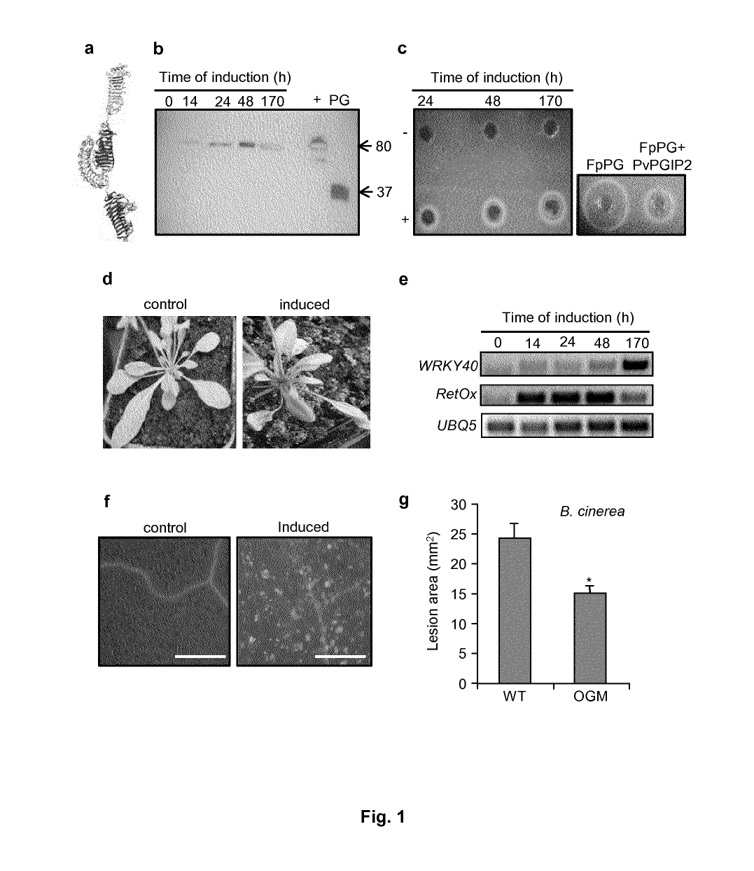

FIGS. 1A-1G (collectively referred to as FIG. 1). Characterisation of the transgenic plants expressing OGM inducible by chemical treatment. (A) Schematic representation of two OGM molecules that interact. PvPGIP2 and FpPG are linked by three alanines and correspond to the N and C terminals of the fusion protein, respectively. (B) Four-week-old plants of a representative transgenic line which express the OGM after one week of induction with β-estradiol. (C) The total protein extracts from the leaves of the rosette (3 μng) of 4-week-old plants of a representative transgenic line which express the OGM after induction with β-estradiol at the times indicated were separated by SDS-PAGE and analysed by means of an immunodecoration assay using the antibody directed against FpPG as the primary one. The purified OGM (+, 80 kDa) and FpPG (PG, 37 kDa) were used as reference proteins. (D) Determination of polygalacturone activity in the protein extracts (3 μg) obtained from leaves of transgenic plants treated, for the times indicated, with DMSO (−) or β-estradiol (+) by means of an agar diffusion assay. (E) Visualisation of callose deposits in the leaves of the rosette of the transgenic plants treated with DMSO (non-induced) or β-estradiol for 170 h by staining with aniline blue. (F) The expression of the genes WRKY40 and RetOx was determined in the transgenic plants treated with β-estradiol for the times indicated by semi-quantitative RT-PCR, using the expression of the gene UBQ5 as reference. (G) Wild plants (WT) and transgenic plants were treated for 170 h with β-estradiol and subsequently inoculated with a suspension of spores of B. cinerea. After two days, the area of the lesion generated by the fungus was measured. The bars indicate the mean lesion area produced by the fungus (n>10). The asterisk indicates a statistically significant difference, in accordance with the Student t-test (P<0.05). This experiment was repeated three times with comparable results.

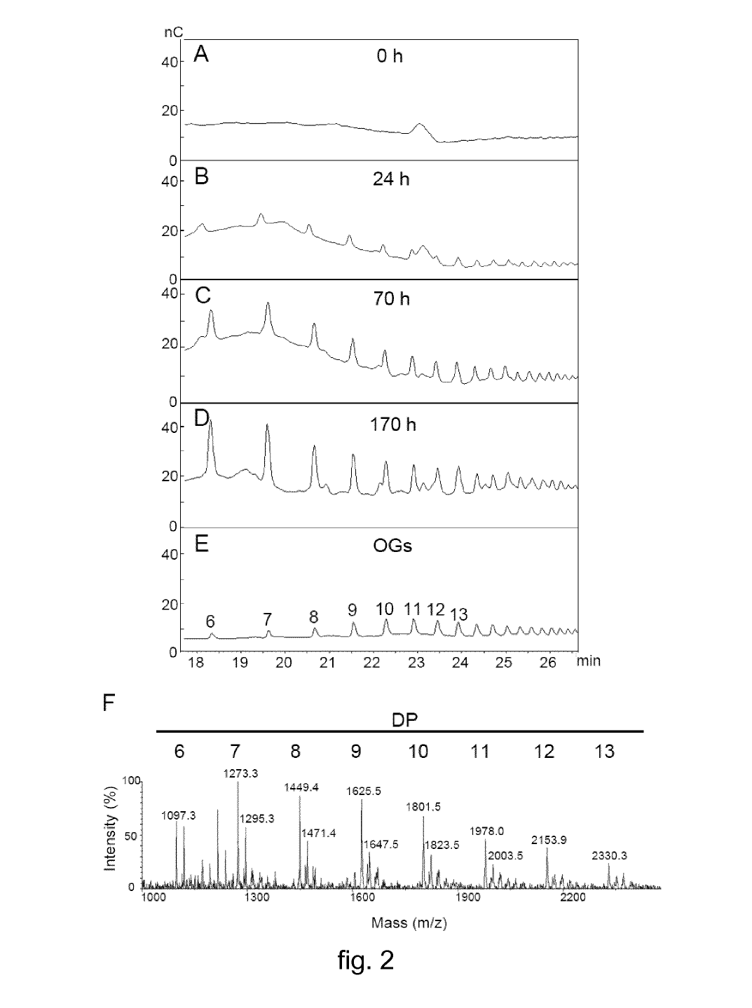

FIGS. 2A-2F (collectively referred to as FIG. 2). Inducible release of oligogalacturonides from plants expressing the OGM. (A-E) HPAEC-PAD analysis on pectin-enriched fractions of cell walls extracted from plants belonging to a representative transgenic line expressing the OGM under the control of a β-estradiol-inducible promoter. Four-week-old plants were treated with the inducer for 0 (A), 24 (B), 70 (C) and 170 hours (D) prior to extraction. A preparation of OGs purified with a degree of polymerisation (DP) of between 6 and 16 was used as a reference (E). The chromatograms show the signal intensity (nC, y axis) as a function of the retention time (minutes, X axis). (F) MALDI-TOF analysis of the same pectin fraction indicated in (D). The numbers indicate the DP of the oligogalacturonides identified as sodium adducts of the same mass as the corresponding peak. The graph shows the intensity of the signals (expressed as a percentage, Y axis) as a function of the mass of the ion (m/z, X axis).

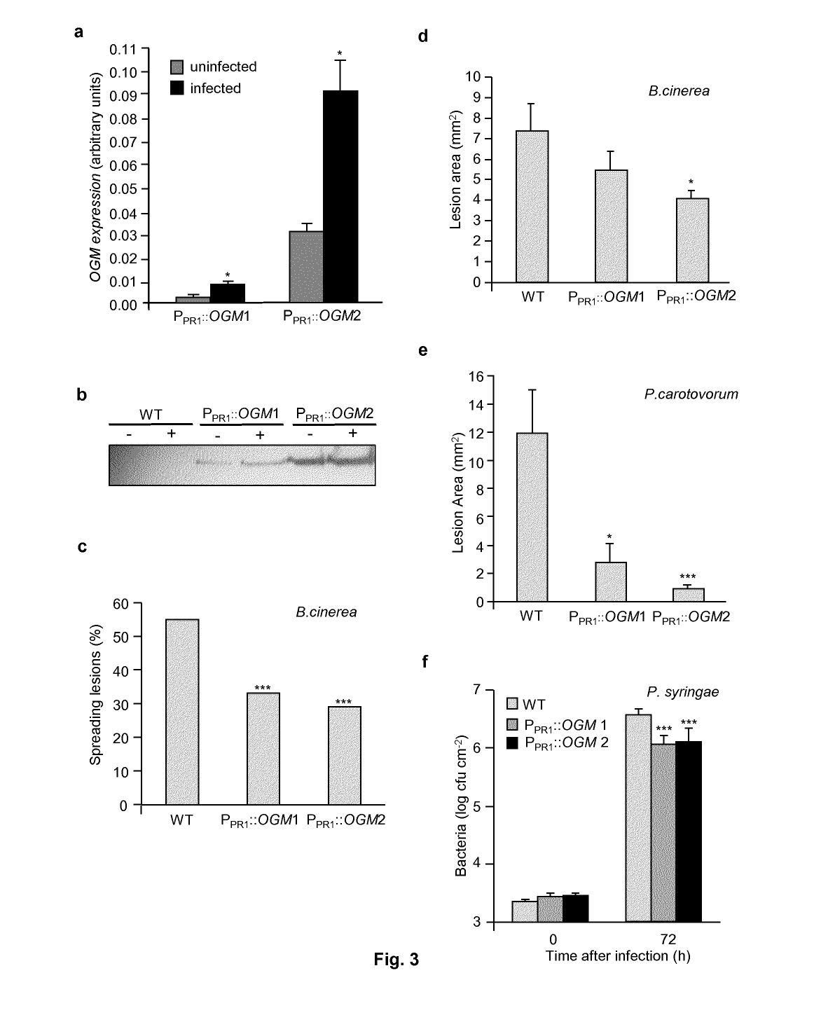

FIGS. 3A-3F (collectively referred to as FIG. 3). Pathogen-inducible expression of the OGM imparts an increased resistance to microbial infections. (A) Four-week-old plants belonging to two independent lines expressing the OGM under the control of the pathogen-inducible promoter which regulates the expression of PR1 (P_{PR1}::OGM 1 and 2) were inoculated with a suspension of spores of B. cinerea and the levels of expression of the transgene were quantified before (grey bars) and two days after infection (black bars) by quantitative PCR, using the expression of the gene UBQ5 as reference. The bars indicate the mean level of expression (in arbitrary units)±SD (n=3). (B) The accumulation of the OGM in leaves of wild type plants (WT) and transgenic plants before (−) and two days after inoculation with B. cinerea (+). The total protein extract (30 μg) was separated by SDS-PAGE and subjected to an immunodecoration assay, using a primary antibody directed against FpPG (C-D) The leaves of the rosette of wild type plants (WT), P_{PR1}::OGM line 1 and line 2 were inoculated with B. cinerea and—after 72 h—a determination was made both of the percentage of infections that took hold, measured as a percentage of inocula that developed grey rot lesions (C) and the mean area of the lesions (D). The bars indicate (C) the mean of three independent experiments (n>12 in each experiment); the bars in (D) indicate the mean area±SE (n>12). (E) The leaves of the rosette of wild type plants (WT), P_{PR1}::OGM line 1 and line 2 were inoculated with P. carototovorum and the area of the lesions was measured after 16 hours. The bars indicate the mean area of the lesions±SE (n>12). (f) The leaves of the rosette of untransformed plants (WT), P_{PR1}::OGM line 1 and line 2 were inoculated with P. syringae pv tomato DC3000 and the bacterial growth within the inoculated tissue was determined at the times indicated. The bars indicate the colony-forming units (cfu) per cm² of leaf tissue (n=6). The asterisks in (D-F) indicate the statistically significant differences between the control plant and the transgenic plants, in accordance with Fischer's exact test (C) or the Student t-test (D-F). *, P<0.05; ***, P<0.01. The experiments in (D-F) were repeated three times with comparable results.

The transgenic plants belonging to both lines were significantly more resistant to infection, showing a 75% reduction in the bacterial load detected in the tissues compared to that observed in wild type plants (WT).

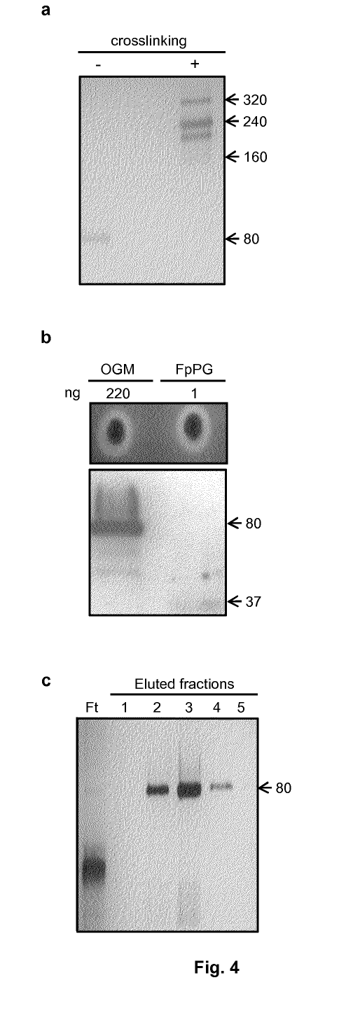

FIGS. 4A-4C (collectively referred to as FIG. 4). Biochemical characterisation of the OGM expressed in P. pastoris. (A) SDS-PAGE analysis (7.5% acrylamide) on the purified fusion protein (OGM) and the one subjected to crosslinking (OGM-cl), where it is possible to detect the formation of multimers ranging from the dimer to the tetramer and the disappearance of the monomer. (B) Top panel, evaluation by agar diffusion assay of the polygalacturone activity carried out by 220 ng of purified OGM and by 1 ng of purified FpPG; bottom panel, immunodecoration analysis of the same protein samples using an antibody directed against the FpPG The expected molecular weights of the OGM (80 kDa) and FpPG (35 kDa) are shown. In the culture filtrate of the untransformed (mock) P. pastoris neither the activity nor the presence of the protein were detected. (C) The fractions eluted by affinity chromatography AnPGII were subjected to SDS-PAGE analysis (10% acrylamide) and visualised by Ag staining. The OGM (80 KDa) was eluted in the fractions. Ft represents the fraction containing the proteins not bound to the column.

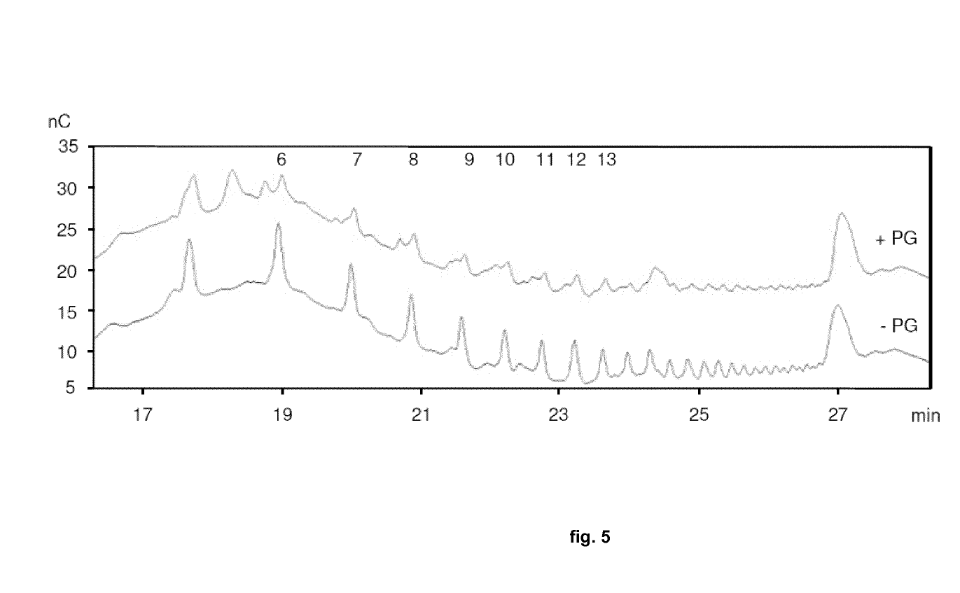

FIG. 5. The OGs released by plants expressing the OGM are hydrolysed by polygalacturonase. A fraction of the cell wall of leaves of the rosette enriched in pectin was extracted from the transgenic plant expressing the OGM under the control of the β-estradiol-inducible promoter 170 hours after the time of induction. The pectic fractions were analysed by HPAEC-PAD before (−) and after treatment with 5 μg of pure FpPG (+). The chromatogram shows the intensity of the signals (nC, Y axis) as a function of the retention times (minutes, X axis).

### EXAMPLE

### Materials and Methods

### Strains

E. coli DH5α [genotype: F-φ80lacZΔM15 Δ(lacZYA-argF)U169 deoR recA1 endA1 hsdR17(rk, mk+) phoA supE44 thi-1 gyrA96 relA1 λ-(Invitrogen)

A. tumefaciens LBA4404 (INVITROGEN, catalogue number: 18313-015)

P. pastoris X33 (wild type) (Invitrogen)

A. thaliana Col-0 (wild type) (purchased from Lehle Seeds)

Construction of the Gene Cassette for the Expression of the Chimeric Fusion Protein PG-PGIP (OGM) in P. pastoris (Corresponding to the Amino Acid Sequence of the OGM Expressed in Pichia (SEQ ID NO:8))

For the expression of the fusion protein in P. pastoris, the 5′ end of the gene coding for PvPGIP2 was fused to the sequence coding for the alpha factor of yeast present in the integrative vector pGAPZ alpha which enables the constitutive expression of the transgene; the construct pGAPZα-PGIP2 was thus obtained. The gene coding for the mature protein PvPGIP2 was amplified by means of specific primers (EcoRIPGIP2Fw (SEQ ID NO: 10) and NotIPGIP2Rv (SEQ ID NO: 11)) which introduced the “EcoRI” and “NotI” restriction sites at the 5′ and 3′ ends, respectively; the gene was then cloned at the multiple cloning site of the vector by exploiting the aforesaid restriction sites. In parallel, “NotI” and “XbaI” restriction sites were introduced at the 5′ and 3′ ends, respectively, of the sequence coding for FpPG using specific primers (NotIFpPGFw (SEQ ID NO: 12) and XbaIFpPGRv (SEQ ID NO: 13)) via PCR. The primer which readapted the SI end of the FpPG gene by inserting the restriction site NotI maintained the correct reading frame between the PvPGIP2 and FpPG and introduced 9 further nucleotide bases, which would code the peptide linker composed of 3 alanines. The fragment coding for FpPG was thus cloned in the multiple cloning site using the “NotI” and “XbaI” restriction sites. The gene fusion (called OG-machine; abbreviated as OGM) was sequenced to exclude the presence of undesirable mutations. The recombinant plasmid was linearised by means of the AvrII restriction enzyme, necessary for site-specific recombination in P. pastoris. The transformation, selection and growth of Pichia took place according to the instructions given in the Invitrogen manual. The filtrates of cultures grown for 3 days were tested both by means of an agar diffusion assay and an immunodecoration assay. The OGM was purified using the same procedure as used to purify PvPGIP2 from a culture filtrate of P. pastoris as described in (29).

Primers used for the construction of gene cassettes coding the OGM for expression in P. pastoris and β-estradiol-inducible expression in A. thaliana:

EcoRIPGIP2Fw:
(SEQ ID NO: 10)
5′ ATCGATGAATTCGAGCTATGCAACCCACA 3′
NotIPGIP2Rv:
(SEQ ID NO: 11)
5′-TCTTCTAAGTGCGGCCGCAGTGCAGGCAGGAAGAG-3′
NotIFpPGFw:
(SEQ ID NO: 12)
5′-TCAACACTATGCGGCCGCACCCTGCTCCGTGACTGAG-3′
XbaIFpPGRv:
(SEQ ID NO: 13)
5′-ATCGATTCTAGACTAGCTGGGGCAAGTGTTG-3′
AvrIISP1Fw:
(SEQ ID NO: 14)
5′-ACTAAGCCTAGGACTATCTAGAATGACTCAATTCAATATCCCAG-3′
EheIPGIP2Rv:
(SEQ ID NO: 15)
5′-GGGGATGGCGCCGGAG-3′
XhoISP1Fw:
(SEQ ID NO: 16)
5′-ACTAAGCTCGAGATGACTCAATTCAATATCCCAG-3′
PacIFpPGRv:
(SEQ ID NO: 17)
5′-CCTAAGTTAATTAACTAGCTGGGGCAAGTGTTG-3′

The underlined sequences indicate the restriction sites introduced.

### Molecular Crosslinking Experiment Conducted on the OGM

1 μg of pure OGM was subjected to auto-crosslinking in a volume of 50 μL of a solution containing 50 mM sodium acetate pH 4.6, to which methanol-free formaldehyde was added at the final concentration of 1% (Thermo-Fisher Scientific, U.S.A). The reaction was incubated at a temperature of 28° C. for 16 hours. 2 μL of the reaction was analysed via SDS-PAGE.

Preparation of the Construct for β-Estradiol-Inducible Expression in A. thaliana

The cDNA coding for the signal peptide of PvPGIP2 (SP), available already fused to PvPGIP2 (27), was amplified up to the EheI restriction site located in the sequence of PvPGIP2 a+550 pairs of bases from the first ATG using the primers called AvrIISP1Fw and EheIPGIP2Rv (SEQ ID NO:14 and SEQ ID NO: 15, respectively). The amplified fragment was cloned in the construct used for expression in P. pastoris using the restriction sites of the AvrII and EheI enzymes. The new construct obtained, which consisted in a fusion between the cDNA coding for the OGM and the sequence coding for the signal peptide for the secretion of PvPGIP2 into the apoplast was thus introduced by means of electroporation in E. coli DH5α for amplification of the plasmid.

The cDNA coding for the fusion protein fused to the signal peptide for the secretion of PvPGIP2 was amplified by PCR using the specific XhoISP1Fw and PacIFpPGRv primers (SEQ ID NO:16 and SEQ ID NO: 17, respectively) which introduced the XhoI and PacI restriction sites at the 5^{I} and 3^{I} ends of the transgene, respectively. The gene readapted to the ends was then cloned in the vector pMDC7 for β-estradiol-inducible expression in plants (31) using the same restriction sites.

The final construct pMDC7.SP-OGM was amplified in E. coli DH5α, and then plasmid extraction took place; the purified plasmid was introduced in A. tumefaciens LBA4404 by electroporation.

Stable Transformation of A. thaliana

The transgenic plants of Arabidopsis thaliana ecotype Col-0 were generated by infection of the flower primordia mediated by A. tumefaciens according to the procedure described in (41). Following the transformation, the transgenic lines were selected in generation T1 by seeding on a plate containing MS solid medium and hygromycin (23 mg L-1) as a positive selection marker. The transgenic lines of the hygromycin-resistant T2 generation were selected for subsequent analyses; in particular, the lines of the T2 generation that showed a segregation of the transgene in a 3:1 ratio were isolated for selection of the transforming homozygote in the T3 generation.

### Induction of Expression in the Transgenic Lines of the T3 Generation Using β-Estradiol

XVE is a transcriptional chimeric factor constitutionally expressed in the nucleus of the plant cell transformed with the T-DNA deriving from the vector pMDC7. XVE is capable of transcribing the transgene regulated by the inducible promoter OlexA-46 only in the presence of β-estradiol (31). The induction of expression was achieved by spraying 1.5 mL of a solution containing 50 μM β-estradiol per transgenic plant.

### Analysis of Gene Expression by Semiquantitative RT-PCR

The removed leaf tissues were frozen in liquid nitrogen, homogenised by means of an MM301 Ball Mill (Retsch), and the total RNA was extracted using Isol-RNA Lysis Reagent (5 Prime), following the instructions provided in the manufacturer's manual. The RNA was treated with RQ1 DNase (PROMEGA) and the first strand of the cDNA was synthesized using the reverse transcript ImProm-II (PROMEGA), in accordance with the manufacturer's instructions. The expression levels of each gene relative to the expression of the gene UBQ5 were determined using a modification of the Pfaffl method (42) as previously described in (43). The (RT)-PCR reaction was conducted in a 50-μL reaction mixture containing 2 μL of cDNA, 1× buffer (RBCBioscience), 3 mm MgCl2, 100 μm of each dNTP, 0.5 μm of each primer (primers EcoRIPGIP2Fw (SEQ ID NO:10) and EheIPGIP2Rv (SEQ ID NO:15)) and 1 unit of Taq polymerase. 25, 30, and 35 amplification cycles were carried out by PCR for every pair of primers in order to verify the linearity of the amplification reaction. The PCR products were separated by agarose gel electrophoresis and visualised by means of ethidium bromide.

### Immunodecoration Assay

The extraction of total proteins from leaf tissue was carried out using a buffer consisting of 20 mM sodium acetate pH 4.6 and 1M NaCl, in which the leaves previously homogenised by means of an MM301 Ball Mill (Retsch) were incubated for 20 minutes. The same quantities of total proteins were separated by SDS-PAGE analysis and then transferred onto a nitrocellulose membrane using, as the transfer buffer, a solution containing 25 Mm TRIS, 192 mM glycine, pH 8.3, and 20% methanol at the temperature of 4° C. for 1 h. Following the transfer, the filter was stained with Ponceau S in order to verify equal loading for all samples; then the filter was saturated by incubating it for 2 h in a solution consisting of 50 mM phosphate buffer, 150 mM NaCl and 3% bovine serum albumin (BSA, SIGMA ALDRICH); subsequently, the filter was incubated for 12 h with a primary antibody directed toward the FpPG (40). After suitable washing, the membrane was incubated with a secondary antibody conjugated to horseradish peroxidase (Amersham, UK) for approximately 2 h. The membrane was washed again and treated with ECL reagents (Amersham, UK) in order to promote the detection of the transgenic protein.

### Determination of Callose Deposition

Leaves of 5-week-old plants were sprayed with a solution containing 50 μM β-estradiol. After 170 h, about 4 leaves were collected from 3 independents plants and dehydrated in a solution consisting of 100% ethanol for about 2 hours. The leaves were then incubated for 15 minutes in 75% ethanol and, finally, in 50% ethanol. Following this pretreatment the leaves were washed in 150 mM phosphate buffer pH 8.0 and then stained for 1 h at 25° C. in 150 mM phosphate buffer, pH 8.0, containing 0.01% (w/v) aniline blue. After staining, the leaves were incubated in 50% glycerol and examined by epifluorescence UV using an Axioskop 2 plus microscope (Zeiss). Pictures were taken with a ProgRes C10 3.3 Megapixel colour digital camera (Jenoptik).

### Infection Assays

Botrytis cinerea was propagated in a solid medium consisting of 20 g l-1 malt extract, 10 g l-1 peptone (Difco, Detroit, USA), and 15 g l-1 agar for 7-10 days at +24° C. with a photoperiod of 12 h prior to collection of the spores. The rosette leaves of Arabidopsis plants were placed on Petri plates containing 0.8% agar, with the petiole inserted into the solid medium to act as a support. Inoculation was carried out by placing on each side of the central vein of each leaf 5 microlitres of a solution consisting of PDB liquid medium (PDB; Difco, Detroit, USA) containing a suspension of 5×105 conidiospores mL-1. The plates were incubated at 22° C. under constant light for 2 days. A high level of humidity was maintained by covering the plates with transparent film. Under the experimental conditions, the majority of the infections produced a rapid expansion of rot lesions of comparable diameter. The size of the lesion was determined by measuring the diameter or, in the case of oval lesions, the major axis of the necrotic area.

P. carotovorum subsp. carotovorum (formerly E. carotovora subsp. carotovora) was obtained from DSMZ GmbH Germany (strain DSMZ 30169). Following growth in Luria-Bertani (LB) liquid medium, the bacteria were suspended in 50 mM potassium phosphate buffer (pH 7.0) and inoculated at a concentration of 5×10⁷ cells mL-1. In each experiment, 12 mature leaves were collected (3 leaves per plant) and placed on damp filter paper in Petri capsules; they were then inoculated with 5 microlitres of the bacterial suspension and maintained at 22° C. and with a photoperiod of 12 hours. The area of the lesions was obtained by measuring the surface of the macerated tissue 16 hours after infection. The areas were measured with ImageJ software (WS Rasband, ImageJ; National Institutes of Health, Bethesda, Md., USA). The experiment was repeated three times with different lots of plants, and a statistical analysis of the results was conducted by unidirectional analysis of variance (ANOVA), followed by the Student-Tukey range test.

Pseudomonas syringae pv tabaci DC3000 was propagated in LB liquid medium at 28° C. for 1 day; the bacterial suspension was resuspended in 10 mM MgCl₂ (1×10⁶ cell/ml). The inoculations were carried out by infiltrating the bacterial suspension using a 1 ml needleless syringe.

### Isolation and Detection of Oligogalacturonides in the Transgenic Plants

Leaves (approximately 100 mg per sample) belonging to transgenic and wild type plants about 4 weeks old were frozen in liquid nitrogen following induction with beta-estradiol and homogenised using a Retschmill machine (model MM200; Retsch) at 25 Hz for 1 min. The pulverised tissue was washed twice using 1 mL of a solution consisting of 70% ethanol and precipitated by centrifugation at 20,000×g for 10 min. The precipitate was then washed twice with a chloroform:methanol mixture (1:1, v/v) and centrifuged at 20000×g for 10 minutes. After centrifugation, the precipitate was suspended twice with acetone and again precipitated by centrifugation at 20000×g for 10 min. The pellet obtained was incubated overnight under the air flow of a chemical fume hood in order to favour evaporation of the solvent, and then resuspended using 200 μL of Ch buffer (composition of the Ch buffer: 50 mM ammonium acetate pH 5, 50 mM CDTA and 50 mM ammonium oxalate) for two hours under stirring at room temperature. The supernatant was recovered after centrifugation at 20000×g for 10 minutes.

### Analysis by High-Performance Anion Exchange Chromatography (HPAEC) Coupled to a Pulsed Amperometric Detector (PAD)

Analysis of the oligogalacturonides was carried out by HPAEC-PAD. The HPAEC system (ICS-3000, Dionex Corporation, Sunnyvale, Calif., USA) was equipped with a CarboPac PA-200 separation column (2 mm ID×250 mm; Dionex Corporation) and a Carbopac PA-200 guard column (2 mm ID×50 mm; Dionex Corporation). A flow of 0.4 mL min-1 was used at a constant temperature of 25° C. The samples, with an injection volume of 25 μL, were separated using a gradient consisting of 0.05 M KOH (A) and 1 M KOAc in 0.05 M KOH (B) according to the following elution program: 0-30 min from 20% B to 80% B, 30-32 min to 100% B. Prior to the injection of each sample, the column was balanced with 90% A and 10% B for 10 min.

### Treatment of Pectin-Enriched Fractions by Means of Exogenous PG

The pectin-enriched fraction extracted from 20 mg of leaf tissue was treated with 5 μg of pure FpPG for 1 hour at a temperature of 37° C. Subsequently, the reaction mixture was subjected to HPAEC-PAD analysis.

Preparation of the Construct for the Expression of the OGM Under the Control of the Inducible Promoter PR-1 in A. thaliana

The primers for preparing the construct for the pathogen-inducible expression of the OGM under the control of the promoter that regulates the expression of the gene PR-1 (AT2G14610, Accession No. UNIPROT Q39187) in A. thaliana are the following (the underlined sequence indicates the restriction site introduced):

XbaISP1Fw:
A:
(SEQ ID NO: 18)
5′-GACTATCTAGAATGACTCAATTCAATATCCC-3′;
HindIIIPR1Fw:
B:
(SEQ ID NO: 19)
5′-GTTAGCA CAAGCTTGTT TTAAC-3′;
XbaIPacIFpPGRv:
C:
(SEQ ID NO: 20)
5′-CCTAAGTCTAGAGGTCTTAATTAACTAGCTGGGG-3′;
HindIIIPGIP2Rv
D:
(SEQ ID NO: 21)
5′-TGCTTAAGCTTGAAGACATGGTTACTGGGATATTGAATTGAGTCA
TTTTTCTAAGTTGATAATGG-3′.

As the starting plasmid, the cloning procedure used the vector pBI121 (Chen P Y, Wang C K, Soong S C, To K Y: Complete sequence of the binary vector pBI121 and its application in cloning T-DNA insertion from transgenic plants. Mol Breeding 2003 11(4): 287-293), in which the PacI restriction site was introduced upstream (−6) of the SacI restriction site, located at the 3′ end of the gene coding for beta-glucuronidase. The gene coding for beta-glucuronidase was then excised via the restriction sites XbaI and PacI. Subsequently, the OGM fused to the signal peptide of PGIP2 was amplified via the primers XbaISP1Fw (SEQ ID NO: 18) and XbaIPacIFpPGRv (SEQ ID NO: 20) which introduced XbaI and PacI at the 5′ and 3′ ends, respectively, of the transgene and cloned in the vector pBI121 from which the gene coding for beta-glucuronidase were previously removed. As a second step, the terminal portion of the promoter which regulates the expression of the gene PR-1 (1300 base pairs of AT2G14610), including the 5′ UTR sequence was amplified from the gDNA of A. thaliana Col-0 using specific primers (HindIIIPR1Fw (SEQ ID NO: 19) and D (SEQ ID NO: 21)) which introduce the restriction sites HindIII at both ends of the amplified product. As a result, the primer of the antisense strand HindIIIPGIP2Rv readapted the 3′ end of the amplicon, introducing a nucleotide tail consisting of the first 39 bases coding for the signal peptide of PGIP2, which is characterised by the HindIII restriction site in its native sequence. The final product obtained was a transcriptional fusion between the last 1300 pairs of bases of the promoter PR-1, the 5′ UTR region of the gene PR-1 and the sequence of the signal peptide of PvPGIP2 coding for the first 13 amino acids downstream of the first methionine. The fragment was cloned using the restriction site HindIII of the plasmid pBI121 (containing the gene OGM), previously digested with HindIII and dephosphorylated by alkaline phosphatase. It is worth noting that the digestion of pBI121 via the enzyme HindIII provokes the excision of a DNA fragment of 900 pairs of bases corresponding to the promoter 35S. The cassette was sequenced both to exclude the presence of undesirable mutations and to verify the correct orientation of the truncated version of the promoter PR-1.

### Results

In order to exploit the potentialities of OGs as generic plant elicitors during defence responses, we engineered a chimeric fusion protein comprising PvPGIP2 (Uniprot Accession Number: P58822.1; SEQ ID NO:4), an inhibitor originating from the common pea (Phaseolus vulgaris) (27,28), and a PG ligand (FpPG) thereof, originating from the fungus Fusarium phyllophilum (SEQ ID NO:2; corresponding to aa.26-aa.373 of the Uniprot sequence Accession Number: Q07181.1)(29). In this manner, the enzyme and its inhibitor will be simultaneously expressed in a stoichiometric ratio of 1:1, which results in an increase, in vivo, in the production of biologically active OGs. In Pichia pastoris and Arabidopsis thaliana, fusion proteins were expressed with linkers consisting of the module Gly4Ser1 repeated from seven to nine times; their dimensions can permit an intramolecular interaction between enzyme and inhibitor. In both organisms these proteins were subjected to proteolytic cleavage and this caused the release of active FpPG; the high residual polygalacturone activity caused severe growth defects in Arabidopsis. This effect was consistent with the one that had previously been observed in transgenic plants which expressed the PG of Aspergillus niger; such plants were not able to grow as a consequence of the high enzymatic activity present in the tissues (30). Subsequently, a fusion protein with a linker of only three alanine residues was generated; it was short enough not to permit intramolecular interactions, but capable of promoting intermolecular interactions between enzymes and inhibitors belonging to different chimeric molecules (FIG. 1a). When expressed in P. pastoris, the fusion protein was recovered as an intact polypeptide of the expected size, indicating a resistance to proteolysis (FIG. 4a). The protein was purified by affinity chromatography using the PGII of A. niger conjugated to a sepharose matrix, which was capable of binding the PGIP domain of the fusion protein (FIG. 5b, bottom panel). The enzymatic activity of the fusion protein was about 220 times lower than the FpPG (FIG. 4b, top panel). This suggested that the enzymatic activity had decreased markedly in the fusion protein due to the intramolecular interactions between the PG domain of one polypeptide and the PGIP domain of the other (FIG. 1a). Molecular crosslinking experiments confirmed this hypothesis and further revealed the ability of the chimera to bring about chain intermolecular interactions ranging from the dimer (˜160 KDa) to the tetramer (320 KDa) (FIG. 4a), which were not observed when the FpPG or PvPGIP2 underwent crosslinking in the absence of the interaction partner (29). The construct PvPGIP2-FpPG was fused to the signal peptide of the bean PvPGIP2 to enable correct secretion in the cell wall (30) and it was later placed under the control of a β-estradiol-inducible promoter for stable expression in Arabidopsis (31). The presence of the protein in the leaves of the rosette was observed in the transgenic plants after 14 h of treatment with β-estradiol and it reached its maximum accumulation at 170 h after treatment (FIG. 1b). The accumulation was associated with the appearance of slight PG activity, which, as already previously demonstrated, is detected following the formation of the PG-PGIP molecular complex (4,6) (FIG. 1b). Adult transgenic plants did not show any obvious morphological defects when grown under normal conditions. However, following treatment with the inducer, the leaves of the transgenic plants showed discoloration and chlorosis starting from 170 h after the treatment (FIG. 1d). Treating the transgenic plants with an inducer also activated the defence responses typically induced following exogenous treatment with OGs, such as the expression of the marker genes for defence responses (RetOx and WRKY40; FIG. 1e) and the accumulation of callose (FIG. 10. Taken as a whole, these results suggested that an accumulation of OGs might take place in β-estradiol-inducible transgenic plants that can be capable of activating defence responses similar to the ones induced as a result of an exogenous OG treatment. Because of this effect, the fusion protein was called “OG machine” (OGM). In order to verify whether the OGM actually caused the accumulation of OGs in the plant, pectin-enriched fractions were extracted from the leaves of the transgenic plants at 0, 24, 70 and 170 h following treatment with β-estradiol. The fractions were then analysed by high-performance anion exchange chromatography coupled with a pulsed amperometric detector (HPAEC-PAD), which revealed the presence of molecules with retention times comparable to those characterizing a mixture of OGs with a degree of polymerization comprised between 5 and 17; the concentration of these molecules increased with increases in the induction times (FIG. 2a-d). MALDI-TOF mass spectrometry also confirmed that these molecules were characterized by molecular masses corresponding to those of oligomers of unsubstituted polygalacturonic acid with a degree of polymerization of between 6 and 13 (FIG. 2f). Treatment of the fractions with a fully active PG of A. niger caused the molecules to disappear, confirming their OG nature (FIG. 5).

Subsequently, we placed the transgene coding for the OGMs under the control of the terminal portion (1300 bps) of the promoter which regulates the expression of the gene PR-1 of Arabidopsis (PPR-1), strongly induced by bacterial and fungal infections (32-35). The construct (PPR1::OGM) was introduced in Arabidopsis and two independent transgenic lines were selected for a more thorough characterization. Neither transgenic plant showed any evident morphological difference compared to the wild plant Col-0 despite having, in the absence of the pathogen, a basal expression of the transgene that was greater in line 2 than in line 1. After inoculation with Botrytis cinerea, a significant increase (approximately 3-fold) was observed in the transcript coding for the OGM in both lines (FIG. 3a). The immunodecoration analysis confirmed the presence of a basal level of OGM in the uninfected plants, as well as the accumulation of the protein during infection with B. cinerea (FIG. 3b). Subsequently, we compared the susceptibility of the transgenic plants expressing the OGM and of the wild type plant Col-0 to some pathogenic microorganisms. The inocula of B. cinerea in the transgenic plants produced a reduced number of infections, whose success was indicated by the typical grey rot lesion (FIG. 3c). Moreover, the average area of the lesions in the plants of line 2 was significantly smaller than the one produced in wild type plants. The lesions produced in line 1 were likewise smaller than those of wild type plants, but this difference was not wholly significant (FIG. 3d). The transgenic plants also showed a marked resistance against the infections produced by Pectobacterium carotovorum (FIG. 3e) and Pseudomonas syringae pv. tomato DC3000. (FIG. 30. In conclusion, the expression of the OGM under the control of a pathogen-inducible promoter seems to promote a non-specific resistance towards both fungal and bacterial pathogens, in which the release of OGs acts as a generic elicitor of defence responses. The OGM is characterised by a low residual enzymatic activity, which was optimal for regulating a controlled release of OGs in vivo and a controlled release of OGs in plants can in turn activate a wide range of defence responses, imparting resistance against pathogenic microorganisms to the plants.

Our research was mainly aimed at investigating the possible biotechnological applications of the OGM. The expression of the OGM under the control of a pathogen-inducible promoter enabled a rapid activation of defences which protected the transgenic plants against three major pathogens of agronomic interest. The present strategy of employing the OGM for the constitution of transgenic plants can be generally effective towards a broad range of pathogens and can represent a technology for protecting farm crops.

The controlled expression of the OGM following induction can be useful not only for engineering resistance, but also for studying the effects of OGs under physiological conditions. OGs activate defence responses on the one hand, and on the other hand they influence plant development and growth, acting like local auxin antagonists (36-40). The role of OGs as regulators of growth and development is mainly based on experiments that use exogenous OG treatments. To what degree OGs accumulate in intact tissues and in the absence of a pathogen and how they act as endogenous regulators of plant growth and development can now be investigated.

### REFERENCES

1. Newman, M. A., Sundelin, T., Nielsen, J. T. & Erbs, G. MAMP (microbe-associated molecular pattern) triggered immunity in plants. Front Plant Sci. 4, 139 (2013).
2. Hahn, M. G., Darvill, A. G. & Albersheim, P. Host-pathogen interactions. XIX. The endogenous elicitor, a fragment of a plant cell wall polysaccharide that elicits phytoalexin accumulation in soybeans. Plant Physiol. 68, 1161-1169 (1981).
3. Ferrari, S., Savatin, D. V., Sicilia, F., Gramegna, G, Cervone, F. & De Lorenzo, G. Oligogalacturonides: plant damage-associated molecular patterns and regulators of growth and development Frontiers in Plant Science 4, 1-16 (2013).
4. Cervone, F., Hahn, M. G., De Lorenzo, G, Darvill, A. & Albersheim, P. Host-pathogen interactions. XXXIII. A plant protein converts a fungal pathogenesis factor into an elicitor of plant defense responses. Plant Physiol. 90, 542-548 (1989).
5. Nothnagel, E. A., McNeil, M., Albersheim, P. & Dell, A. Host-pathogen interactions. XXII. A galacturonic acid oligosaccharide from plant cell walls elicits phytoalexins. Plant Physiol. 71, 916-926 (1983).
6. Cervone, F., De Lorenzo, G., Degrà, L., Salvi, G. & Bergami, M. Purification and characterization of a polygalacturonase-inhibiting protein from Phaseolus vulgaris L. Plant Physiol. 85, 631-637 (1987).
7. Ferrari, S., Vairo, D., Ausubel, F. M., Cervone, F. & De Lorenzo, G. Tandemly duplicated Arabidopsis genes that encode polygalacturonase-inhibiting proteins are regulated coordinately by different signal transduction pathways in response to fungal infection. Plant Cell 15, 93-106 (2003).
8. Powell, A. L., van Kan, J., ten Have, A., Visser, J., Greve, L. C., Bennett, A. B., Labavitch, J. M. Transgenic expression of pear PGIP in tomato limits fungal colonization. Mol. Plant-Microbe Interact. 13, 942-950 (2000).
9. Manfredini, C., Sicilia, F., Ferrari, F., Pontiggia, D., Salvi, G., Caprari, C., Lorito, M., De Lorenzo, G. Polygalacturonase-inhibiting protein 2 of Phaseolus vulgaris inhibits BcPG1, a polygalacturonase of Botrytis cinerea important for pathogenicity, and protects transgenic plants from infection. Physiol. Mol. Plant Pathol. 67, 108-115 (2005).
10. D'Ovidio, R., Raiola, A., Capodicasa, C., Devoto, A., Pontiggia, D., Roberti, S., Galletti, R., Conti, E., O'Sullivan, D., De Lorenzo, G Characterization of the complex locus of bean encoding polygalacturonase-inhibiting proteins reveals subfunctionalization for defense against fungi and insects. Plant Physiol. 135, 2424-2435 (2004).
11. Casasoli, M., Federici, L., Spinelli, F., of Matteo, A., Vella, N., Scaloni, F., Fernandez-Recio, J., Cervone, F. & De Lorenzo, G. Integration of evolutionary and desolvation energy analysis identifies functional sites in a plant immunity protein Proc. Natl. Acad. Sci. USA 106, 7666-7671(2009).
12. Davis, K. R., Darvill, A. G., Albersheim, P. & Dell, A. Host-pathogen interactions. XXIX. Oligogalacturonides released from sodium polypectate by endopolygalacturonic acid lyase are elicitors of phytoalexins in soybean. Plant Physiol. 80, 568-577 (1986).
13. Walker-Simmons, M., Hadwiger, L. & Ryan, C. A. Chitosans and pectic polysaccharides both induce the accumulation of the antifungal phytoalexin pisatin in pea pods and antinutrient proteinse inhibitors in tomato leaves. Biochem. Biophys. Res. Commun. 110, 194-199 (1983).
14. Jin, D. F. & West, C. A. Characteristics of galacturonic acid oligomers as elicitors of casbene synthetase activity in castor bean seedlings. Plant Physiol. 74, 989-992 (1984).
15. Davis, K. R. & Hahlbrock, K. Induction of defense responses in cultured parsley cells by plant cell wall fragments. Plant Physiol. 85, 1286-1290 (1987).
16. Broekaert, W. F. & Peumans, W. J. Pectic polysaccharides elicit chitinase accumulation in tobacco. Physiol. Plant. 74, 740-744 (1988).
17. Denoux, C., Galletti, R., Mammarella, N., Gopalan, S., Werck, D., De Lorenzo, G., Ferrari, S., Ausubel, F. M. & Dewdney, J. Activation of defense response pathways by OGs and Flg22 elicitors in Arabidopsis seedlings Mol Plant 1, 423-445 (2008).
18. Galletti, R., De Lorenzo, G. & Ferrari, S. Host-derived signals activate plant innate immunity. Plant Signal. Behav. 4, 33-34 (2009).
19. Bellincampi, D., Dipierro, N., Salvi, G., Cervone, F. & De Lorenzo, G. Extracellular H₂O₂ induced by oligogalacturonides is not involved in the inhibition of the auxin-regulated rolB gene expression in tobacco leaf explants. Plant Physiol. 122, 1379-1385 (2000).
20. Galletti, R., Denoux, C., Gambetta, S., Dewdney, J., Ausubel, F. M., De Lorenzo, G. & Ferrari, S. The AtrbohD-mediated oxidative burst elicited by oligogalacturonides in Arabidopsis is dispensable for the activation of defense responses effective against Botrytis cinerea Plant Physiol. 148, 1695-1706 (2008).
21. Brutus, A., Sicilia, F., Macone, A., Cervone, F. & De Lorenzo, G. A domain swap approach reveals a role of the plant wall-associated kinase 1 (WAK1) as a receptor of oligogalacturonides. Proc. Natl. Acad. Sci. USA 107, 9452-9457 (2010).
22. De Lorenzo, G., Brutus, A., Savatin, D. V., Sicilia, F. & Cervone, F. Engineering plant resistance by constructing chimeric receptors that recognize damage-associated molecular patterns (DAMPs). FEBS Lett. 585, 1521-1528 (2011).
23. Ferrari, S., Galletti, R., Denoux, C., De Lorenzo, G, Ausubel, F. M. & Dewdney, J. Resistance to Botrytis cinerea induced in Arabidopsis by elicitors is independent of salicylic acid, ethylene, or jasmonate signaling but requires PHYTOALEXIN DEFICIENT3 Plant Physiol. 144, 367-379 (2007).
24. Galletti, R., Ferrari, S. & De Lorenzo, G. Arabidopsis MPK3 and MPK6 play different roles in basal and oligogalacturonide- or flagellin-induced resistance against Botrytis cinerea. Plant Physiol 157, 804-814 (2011).
25. Termeer, C., Benedix, F., Sleeman, J., Fieber, C., Voith, U., Ahrens, T., Miyake, K., Freudenberg, M., Galanos, C. & Simon, J. C. Oligosaccharides of Hyaluronan activate dendritic cells via toll-like receptor 4 J. Exp. Med. 195, 99-111 (2002).
26. Nurnberger, T., Brunner, F., Kemmerling, B. & Piater, L. Innate immunity in plants and animals: striking similarities and obvious differences. Immunol. Rev. 198, 249-266 (2004).
27. Leckie, F., Maffei, B., Capodicasa, C., Hemmings, A., Nuss, L., Aracri, B., De Lorenzo, G. & Cervone, F. The specificity of polygalacturonase-inhibiting protein (PGIP): a single amino acid substitution in the solvent-exposed b-strand/b-turn region of the leucine-rich repeats (LRRs) confers a new recognition capability EMBO J. 18, 2352-2363 (1999).
28. Farina, A., Rocchi, V., Janni, M., Benedettelli, S., De Lorenzo, G., D'Ovidio, R. The bean polygalacturonase inhibiting protein 2 (PvPGIP2) is highly conserved in common bean (Phaseolus vulgaris L.) germplasm and related species. Theor. Appl. Genet. 118, 1371-1379 (2009).
29. Benedetti, M., Leggio, C., Federici, L., De Lorenzo, G., Pavel, N. V. & Cervone, F. Structural Resolution of the Complex between a Fungal Polygalacturonase and a Plant Polygalacturonase-Inhibiting Protein by Small-Angle X-Ray Scattering Plant Physiol. 157, 599-607 (2011).
30. Capodicasa, C., Vairo, D., Zabotina, O., McCartney, L., Caprari, C., Maffei, B., Manfredini, C., Aracri, B., Benen, J., Knox, P., De Lorenzo, G., Cervone, F. Targeted modification of homogalacturonan by transgenic expression of a fungal polygalacturonase alters plant growth. Plant Physiol. 135, 1294-1304 (2004).
31 Zuo, J., Niu, Q. W. & Chua, N. H. Technical advance: An estrogen receptor-based transactivator XVE mediates highly inducible gene expression in transgenic plants. Plant J. 24, 265-273 (2000).
32. Cao, H., Bowling, S. A., Gordon, A. S. & Dong, X. Characterization of an Arabidopsis mutant that is nonresponsive to inducers of systemic acquired resistance. Plant Cell 6, 1583-1592 (1994).
33. Zhao, Y, Thilmony, R., Bender, C. L., Schaller, A., He, S. Y. and Howe, G. A. Virulence systems of Pseudomonas syringae pv. tomato promote bacterial speck disease in tomato by targeting the jasmonate signaling pathway. Plant J. 36, 485-499 (2003).
34. Lee, M. W. & Yang, Y. Transient expression assay by agroinfiltration of leaves. Methods Mol. Biol. 323, 225-229 (2006).
35. Robert-Seilaniantz, A., Grant, M. & Jones, J. D. G. Hormone Crosstalk in Plant Disease and Defense: More Than Just JASMONATE-SALICYLATE Antagonism. Annual Review of Phytopathology, Vol 49 49, 317-343 (2011).
36. Branca, C., De Lorenzo, G. & Cervone, F. Competitive inhibition of the auxin-induced elongation by a-D-oligogalacturonides in pea stem segments. Physiol. Plant. 72, 499-504 (1988).
37. Hayashi, T. & Yoshida, K. Cell expansion and single-cell separation induced by colchicine in suspension-cultured soybean cells. Proc. Natl. Acad. Sci. USA 85, 2618-2622 (1988).
38. Baldwin, E. A. & Pressey, R. Pectic enzymes in pectolyase. Separation, characterization, and induction of ethylene in fruits. Plant Physiol. 90, 191-196 (1989).
39. Filippini, F., Terzi, M., Cozzani, F., Vallone, D. & the Schiavo, F. Modulation of auxin-binding proteins in cell suspensions. II. Isolation and initial characterization of carrot cell variants impaired in somatic embryogenesis. Theor. Appl. Genet. 84, 430-434 (1992).
40. Peretto, R., Favaron, F., Bettini, V., De Lorenzo, G., Marini, S., Alghisi, P., Cervone, F., Bonfante, P. Expression and localization of polygalacturonase during the outgrowth of lateral roots in Allium porrum L. Planta. 188, 164-172 (1992).
41. Clough, S. J. & Bent, A. F. Floral dip: a simplified method for Agrobacterium-mediated transformation of Arabidopsis thaliana. Plant J. 16, 735-43 (1998).
42. Pfaffl, M. W. A new mathematical model for relative quantification in real-time RT-PCR. Nucleic Acids Res. 29, e45 (2001).
43. Ferrari, S., Galletti, R., Vairo, D., Cervone, F. & De Lorenzo, G. Antisense expression of the Arabidopsis thaliana AtPGIP1 gene reduces polygalacturonase-inhibiting protein accumulation and enhances susceptibility to Botrytis cinerea. Mol. Plant Microbe Interact. 19, 931-936 (2006).

1. A nucleic acid molecule coding for a chimeric protein comprising:
an amino acid sequence with a polygalacturonase inhibitor (PGIP) activity of plant origin; and
an amino acid sequence with a polygalacturonase (PG) activity of fungal, bacterial or insect origin.

2. The nucleic acid molecule according to claim 1, wherein said chimeric protein comprises the amino acid sequence with a polygalacturonase inhibitor (PGIP) activity of plant origin at an N-terminal portion, and the amino acid sequence with a polygalacturonase (PG) activity of fungal, bacterial or insect origin at a C-terminal portion.

3. The nucleic acid molecule coding for a chimeric protein according to claim 1, wherein the amino acid sequence with the PGIP activity comprises a sequence selected from the group consisting of:
the sequence of PGIP2 of Phaseolus vulgari (Pv PGIP2) comprising the sequence SEQ ID NO:4;
the sequence of PGIP1 of Phaseolus vulgari (Pv PGIP1) comprising the sequence SEQ ID NO:23;
the sequence of PGIP3 of Phaseolus vulgari (Pv PGIP3) comprising the sequence SEQ ID NO:25;
the sequence of the PGIP of Malus domestica comprising the sequence SEQ ID NO: 26;
the sequence of PGIP1 of Vitis vinifera comprising the sequence SEQ ID NO: 28;
the sequence of PGIP1 of Arabidopsis thaliana comprising the sequence SEQ ID NO:30; the sequence of PGIP2 of Arabidopsis thaliana comprising the sequence SEQ ID NO:31; or
a functional fragment, isoform, or a functional equivalent, variant, mutant, derivative, synthetic, or recombinant functional analogue thereof.

4. The nucleic acid molecule coding for a chimeric protein according claim 1, wherein the amino acid sequence with the PG activity comprises a sequence selected from the group consisting of:
the sequence of PG of Fusarium phyllophilum (FpPG) comprising the sequence SEQ ID NO:2 or SEQ ID NO:22;
the sequence of PG2 of Aspergillus niger comprising the sequence SEQ ID NO:24;
the sequence of the PG of Colletotrichum lupini comprising the sequence SEQ ID NO:27;
the sequence of BcPG2 of Botrytis cinerea comprising the sequence SEQ ID NO:29; or
a functional fragment, isoform, or a functional equivalent, variant, mutant, derivative, synthetic, or recombinant functional analogue thereof.

5. (canceled)

6. (canceled)

7. (canceled)

8. (canceled)

9. (canceled)

10. (canceled)

11. (canceled)

12. The nucleic acid molecule according to claim 1, further comprising a region coding for a linker.

13. The nucleic acid molecule according to claim 1 wherein the linker comprises the sequence Ala, Ala, Ala.

14. The nucleic acid molecule according to claim 1, comprising a region coding for a signal peptide.

15. The nucleic acid molecule according to claim 1, comprising the nucleotide sequence having essentially the SEQ ID NO: 5, SEQ ID NO: 7 or SEQ ID NO: 9.

16. The nucleic acid molecule according to claim 1, further comprising a promoter which is active in plants.

17. The nucleic acid molecule according to claim 16, wherein the promoter regulates the expression of PR-1 gene of Arabidopsis (PPR-1).

18. The nucleic acid molecule of claim 1, incorporated in an expression vector.

19. The expression vector according to claim 18, wherein the nucleic acid molecule is under the control of a promoter which is active in plants.

20. The expression vector according to claim 19, wherein the promoter is pathogen inducible.

21. A method for producing at least one transgenic plant cell, comprising: exposing at least one plant cell to the expression vector of claim 18.

22. (canceled)

23. (canceled)

24. A chimeric protein comprising an amino acid sequence with a polygalacturonase inhibitor (PGIP) activity of plant origin; and an amino acid sequence with a polygalacturonase (PG) activity of fungal, bacterial or insect origin.

25. The chimeric protein according to claim 24, comprising the amino acid sequence having essentially the SEQ ID NO: 6 or the SEQ ID NO: 8 or functional fragment, equivalent, variant, mutant, derivative, synthetic or recombinant functional analogue thereof.

26. A host cell comprising an expression vector, the expression vector comprising:
a nucleic acid molecule coding for a chimeric protein comprising: an amino acid sequence with a polygalacturonase inhibitor (PGIP) activity of plant origin; and an amino acid sequence with a polygalacturonase (PG) activity of fungal, bacterial or insect origin.

27. The nucleic acid molecule according to claim 16, wherein is the promoter is pathogen inducible.

28. The nucleic acid of claim 1 incorporated in at least one host cell.

29. The host cell of claim 27, wherein the host cell is a plant cell.


SEQUENCE LISTING (31 sequences)

SEQ ID NO: 1 - DNA, 1047 bp, Fusarium phyllophilum
ccctgctccgtgactgagtactctggcctcgccaccgctgtctcatcctgcaaaaacatcgtgctcaacggtttccaagtcccgacaggcaagcaactcgacctatccagcctccagaatgactcgaccgttaccttcaagggcacgaccacttttgccaccactgctgataacgactttaatcctatcgtcattagtggaagtaacatcactatcactggtgcatctggccatgtcattgatggcaacggtcaggcgtactgggatggcaaaggttctaacagcaatagcaaccaaaagcccgatcacttcatcgttgttcagaagaccaccggcaactcaaagatcacaaacctaaatatccagaactggcccgttcactgcttcgacattacaggcagctcgcaattgaccatctcagggcttattcttgataacagagctggcgacaagcctaacgccaagagcggtagcttgcccgctgcgcataacaccgacggtttcgacatctcgtccagtgaccacgttacgctggataacaatcatgtttataaccaagatgattgtgttgctgttacttccggtacaaacatcgtcgtttctaacatgtattgctccggcggccatggtcttagtatcggatctgttggtggaaagagcgacaatgtcgttgatggtgttcagttcttgagctcgcaggttgtgaacagtcagaatggatgtcgcatcaagtccaactctggcgcaactggcacgatcaacaacgtcacctaccagaacattgctctcaccaacatcagcacgtacggtgtcgatgttcagcaggactatctcaacggcggccctactggaaagccgaccaacggagtcaagatcagcaacatcaagttcatcaaggtcactggcactgtggctagctctgcccaggattggtttattctgtgtggtgatggtagctgctctggatttaccttctctggaaacgctattactggtggtggcaagactagcagctgcaactatcctaccaacacttgccccagctag

SEQ ID NO: 2 - protein, 348 aa, Fusarium phyllophilum
PCSVTEYSGLATAVSSCKNIVLNGFQVPTGKQLDLSSLQNDSTVTFKGTTTFATTADNDFNPIVISGSNITITGASGHVIDGNGQAYWDGKGSNSNSNQKPDHFIVVQKTTGNSKITNLNIQNWPVHCFDITGSSQLTISGLILDNRAGDKPNAKSGSLPAAHNTDGFDISSSDHVTLDNNHVYNQDDCVAVTSGTNIVVSNMYCSGGHGLSIGSVGGKSDNVVDGVQFLSSQVVNSQNGCRIKSNSGATGTINNVTYQNIALTNISTYGVDVQQDYLNGGPTGKPTNGVKISNIKFIKVTGTVASSAQDWFILCGDGSCSGFTFSGNAITGGGKTSSCNYPTNTCPS

SEQ ID NO: 3 - DNA, 1026 bp, Phaseolus vulgaris
atgactcaattcaatatcccagtaaccatgtcttcaagcttaagcataattttggtcattcttgtatctttgagcactgcacactcagagctatgcaacccacaagacaagcaagcccttctccaaatcaagaaagaccttggcaacccaaccactctctcctcatggcttccaaccaccgactgttgcaacagaacctggctaggtgttttatgcgacaccgacacccaaacatatcgcgtcaacaacctcgacctctccggccttaacctcccaaaaccctaccctatcccttcctccctcgccaacctcccctacctcaattttctatacattggtggcatcaataacctcgtcggtccaatcccccccgccatcgctaaactcacccaactccactatctctatatcacccacaccaatgtctccggcgcaatacccgatttcttgtcacagatcaaaaccctcgtcaccctcgacttctcctacaacgccctctccggcaccctacctccctccatctcttctctccccaacctcgtcggaatcacattcgacggcaaccgaatctccggcgccatccccgactcctacggctcattttcgaagctgttcacgtcgatgaccatctcccgcaaccgcctcaccgggaagattccgccgacgtttgcgaatctgaacctggcgttcgttgacttgtctcgaaacatgctggagggtgacgcgtcggtgttgttcggatcagataagaacacgcagaagatacatctggcgaagaactctcttgcctttgatttggggaaagtggggttgtcaaagaacttgaacgggttggatctgaggaacaaccgtatctatgggacgctaccgcagggactgacgcagctaaagtttctgcacagtttaaatgtgagcttcaacaatctgtgcggtgagattcctcaaggtgggaacttgcaaagatttgacgtttctgcttatgccaacaacaagtgcttgtgtggttctcctcttcctgcctgcact

SEQ ID NO: 4 - protein, 342 aa, Phaseolus vulgaris
MTQFNIPVTMSSSLSIILVILVSLSTAHSELCNPQDKQALLQIKKDLGNPTTLSSWLPTTDCCNRTWLGVLCDTDTQTYRVNNLDLSGLNLPKPYPIPSSLANLPYLNFLYIGGINNLVGPIPPAIAKLTQLHYLYITHTNVSGAIPDFLSQIKTLVTLDFSYNALSGTLPPSISSLPNLVGITFDGNRISGAIPDSYGSFSKLFTSMTISRNRLTGKIPPTFANLNLAFVDLSRNMLEGDASVLFGSDKNTQKIHLAKNSLAFDLGKVGLSKNLNGLDLRNNRIYGTLPQGLTQLKFLHSLNVSFNNLCGEIPQGGNLQRFDVSAYANNKCLCGSPLPACT

SEQ ID NO: 5 - DNA, 2082 bp, Artificial Sequence
  nucleotide sequence of OGM expressed in plant
atgactcaattcaatatcccagtaaccatgtcttcaagcttaagcataattttggtcattcttgtatctttgagcactgcacactcagagctatgcaacccacaagacaagcaagcccttctccaaatcaagaaagaccttggcaacccaaccactctctcctcatggcttccaaccaccgactgttgcaacagaacctggctaggtgttttatgcgacaccgacacccaaacatatcgcgtcaacaacctcgacctctccggccttaacctcccaaaaccctaccctatcccttcctccctcgccaacctcccctacctcaattttctatacattggtggcatcaataacctcgtcggtccaatcccccccgccatcgctaaactcacccaactccactatctctatatcacccacaccaatgtctccggcgcaatacccgatttcttgtcacagatcaaaaccctcgtcaccctcgacttctcctacaacgccctctccggcaccctacctccctccatctcttctctccccaacctcgtcggaatcacattcgacggcaaccgaatctccggcgccatccccgactcctacggctcattttcgaagctgttcacgtcgatgaccatctcccgcaaccgcctcaccgggaagattccgccgacgtttgcgaatctgaacctggcgttcgttgacttgtctcgaaacatgctggagggtgacgcgtcggtgttgttcggatcagataagaacacgcagaagatacatctggcgaagaactctcttgcctttgatttggggaaagtggggttgtcaaagaacttgaacgggttggatctgaggaacaaccgtatctatgggacgctaccgcagggactgacgcagctaaagtttctgcacagtttaaatgtgagcttcaacaatctgtgcggtgagattcctcaaggtgggaacttgcaaagatttgacgtttctgcttatgccaacaacaagtgcttgtgtggttctcctcttcctgcctgcactgcggccgcaccctgctccgtgactgagtactctggcctcgccaccgctgtctcatcctgcaaaaacatcgtgctcaacggtttccaagtcccgacaggcaagcaactcgacctatccagcctccagaatgactcgaccgttaccttcaagggcacgaccacttttgccaccactgctgataacgactttaatcctatcgtcattagtggaagtaacatcactatcactggtgcatctggccatgtcattgatggcaacggtcaggcgtactgggatggcaaaggttctaacagcaatagcaaccaaaagcccgatcacttcatcgttgttcagaagaccaccggcaactcaaagatcacaaacctaaatatccagaactggcccgttcactgcttcgacattacaggcagctcgcaattgaccatctcagggcttattcttgataacagagctggcgacaagcctaacgccaagagcggtagcttgcccgctgcgcataacaccgacggtttcgacatctcgtccagtgaccacgttacgctggataacaatcatgtttataaccaagatgattgtgttgctgttacttccggtacaaacatcgtcgtttctaacatgtattgctccggcggccatggtcttagtatcggatctgttggtggaaagagcgacaatgtcgttgatggtgttcagttcttgagctcgcaggttgtgaacagtcagaatggatgtcgcatcaagtccaactctggcgcaactggcacgatcaacaacgtcacctaccagaacattgctctcaccaacatcagcacgtacggtgtcgatgttcagcaggactatctcaacggcggccctactggaaagccgaccaacggagtcaagatcagcaacatcaagttcatcaaggtcactggcactgtggctagctctgcccaggattggtttattctgtgtggtgatggtagctgctctggatttaccttctctggaaacgctattactggtggtggcaagactagcagctgcaactatcctaccaacacttgccccagctag

SEQ ID NO: 6 - protein, 693 aa, Artificial Sequence
  amino acid sequence of OGM expressed in plant
MTQFNIPVTMSSSLSIILVILVSLSTAHSELCNPQDKQALLQIKKDLGNPTTLSSWLPTTDCCNRTWLGVLCDTDTQTYRVNNLDLSGLNLPKPYPIPSSLANLPYLNFLYIGGINNLVGPIPPAIAKLTQLHYLYITHTNVSGAIPDFLSQIKTLVTLDFSYNALSGTLPPSISSLPNLVGITFDGNRISGAIPDSYGSFSKLFTSMTISRNRLTGKIPPTFANLNLAFVDLSRNMLEGDASVLFGSDKNTQKIHLAKNSLAFDLGKVGLSKNLNGLDLRNNRIYGTLPQGLTQLKFLHSLNVSFNNLCGEIPQGGNLQRFDVSAYANNKCLCGSPLPACTAAAPCSVTEYSGLATAVSSCKNIVLNGFQVPTGKQLDLSSLQNDSTVTFKGTTTFATTADNDFNPIVISGSNITITGASGHVIDGNGQAYWDGKGSNSNSNQKPDHFIVVQKTTGNSKITNLNIQNWPVHCFDITGSSQLTISGLILDNRAGDKPNAKSGSLPAAHNTDGFDISSSDHVTLDNNHVYNQDDCVAVTSGTNIVVSNMYCSGGHGLSIGSVGGKSDNVVDGVQFLSSQVVNSQNGCRIKSNSGATGTINNVTYQNIALTNISTYGVDVQQDYLNGGPTGKPTNGVKISNIKFIKVTGTVASSAQDWFILCGDGSCSGFTFSGNAITGGGKTSSCNYPTNTCPS

SEQ ID NO: 7 - DNA, 1995 bp, Artificial Sequence
  nucleotide sequence of OGM expressed in Pichia
gagctatgcaacccacaagacaagcaagcccttctccaaatcaagaaagaccttggcaacccaaccactctctcctcatggcttccaaccaccgactgttgcaacagaacctggctaggtgttttatgcgacaccgacacccaaacatatcgcgtcaacaacctcgacctctccggccttaacctcccaaaaccctaccctatcccttcctccctcgccaacctcccctacctcaattttctatacattggtggcatcaataacctcgtcggtccaatcccccccgccatcgctaaactcacccaactccactatctctatatcacccacaccaatgtctccggcgcaatacccgatttcttgtcacagatcaaaaccctcgtcaccctcgacttctcctacaacgccctctccggcaccctacctccctccatctcttctctccccaacctcgtcggaatcacattcgacggcaaccgaatctccggcgccatccccgactcctacggctcattttcgaagctgttcacgtcgatgaccatctcccgcaaccgcctcaccgggaagattccgccgacgtttgcgaatctgaacctggcgttcgttgacttgtctcgaaacatgctggagggtgacgcgtcggtgttgttcggatcagataagaacacgcagaagatacatctggcgaagaactctcttgcctttgatttggggaaagtggggttgtcaaagaacttgaacgggttggatctgaggaacaaccgtatctatgggacgctaccgcagggactgacgcagctaaagtttctgcacagtttaaatgtgagcttcaacaatctgtgcggtgagattcctcaaggtgggaacttgcaaagatttgacgtttctgcttatgccaacaacaagtgcttgtgtggttctcctcttcctgcctgcactgcggccgcaccctgctccgtgactgagtactctggcctcgccaccgctgtctcatcctgcaaaaacatcgtgctcaacggtttccaagtcccgacaggcaagcaactcgacctatccagcctccagaatgactcgaccgttaccttcaagggcacgaccacttttgccaccactgctgataacgactttaatcctatcgtcattagtggaagtaacatcactatcactggtgcatctggccatgtcattgatggcaacggtcaggcgtactgggatggcaaaggttctaacagcaatagcaaccaaaagcccgatcacttcatcgttgttcagaagaccaccggcaactcaaagatcacaaacctaaatatccagaactggcccgttcactgcttcgacattacaggcagctcgcaattgaccatctcagggcttattcttgataacagagctggcgacaagcctaacgccaagagcggtagcttgcccgctgcgcataacaccgacggtttcgacatctcgtccagtgaccacgttacgctggataacaatcatgtttataaccaagatgattgtgttgctgttacttccggtacaaacatcgtcgtttctaacatgtattgctccggcggccatggtcttagtatcggatctgttggtggaaagagcgacaatgtcgttgatggtgttcagttcttgagctcgcaggttgtgaacagtcagaatggatgtcgcatcaagtccaactctggcgcaactggcacgatcaacaacgtcacctaccagaacattgctctcaccaacatcagcacgtacggtgtcgatgttcagcaggactatctcaacggcggccctactggaaagccgaccaacggagtcaagatcagcaacatcaagttcatcaaggtcactggcactgtggctagctctgcccaggattggtttattctgtgtggtgatggtagctgctctggatttaccttctctggaaacgctattactggtggtggcaagactagcagctgcaactatcctaccaacacttgccccagctag

SEQ ID NO: 8 - protein, 664 aa, Artificial Sequence
  amino acid sequence of OGM expressed in Pichia
ELCNPQDKQALLQIKKDLGNPTTLSSWLPTTDCCNRTWLGVLCDTDTQTYRVNNLDLSGLNLPKPYPIPSSLANLPYLNFLYIGGINNLVGPIPPAIAKLTQLHYLYITHTNVSGAIPDFLSQIKTLVTLDFSYNALSGTLPPSISSLPNLVGITFDGNRISGAIPDSYGSFSKLFTSMTISRNRLTGKIPPTFANLNLAFVDLSRNMLEGDASVLFGSDKNTQKIHLAKNSLAFDLGKVGLSKNLNGLDLRNNRIYGTLPQGLTQLKFLHSLNVSFNNLCGEIPQGGNLQRFDVSAYANNKCLCGSPLPACTAAAPCSVTEYSGLATAVSSCKNIVLNGFQVPTGKQLDLSSLQNDSTVTFKGTTTFATTADNDFNPIVISGSNITITGASGHVIDGNGQAYWDGKGSNSNSNQKPDHFIVVQKTTGNSKITNLNIQNWPVHCFDITGSSQLTISGLILDNRAGDKPNAKSGSLPAAHNTDGFDISSSDHVTLDNNHVYNQDDCVAVTSGTNIVVSNMYCSGGHGLSIGSVGGKSDNVVDGVQFLSSQVVNSQNGCRIKSNSGATGTINNVTYQNIALTNISTYGVDVQQDYLNGGPTGKPTNGVKISNIKFIKVTGTVASSAQDWFILCGDGSCSGFTFSGNAITGGGKTSSCNYPTNTCPS

SEQ ID NO: 9 - DNA, 3373 bp, Artificial Sequence
  nucleotide sequence of OGM fused to promoter PR-1
aagcttgttttaacttataaaatgattctccctccatataaaaaagtttgattttatagaatgtttataccgattaaaaaaataataatgcttagttataaattactatttattcatgctaaactatttctcgtaactattaaccaatagtaattcatcaaattttaaaattctcaattaattgattcttgaaattcataaccttttaatattgattgataaaaatatacataaactcaatctttttaatacaaaaaaactttaaaaaatcaatttttctgattcggagggagtatatgttattgcttagaatcacagattcatatcaggattggaaaattttaaagccagtgcatatcagtagtcaaaattggtaaatgatatacgaaggcggtacaaaattaggtatactgaagatagaagaacacaaaagtagatcggtcacctagagtttttcaatttaaactgcgtattagtgtttggaaaaaaaaaacaaagtgtatacaatgtcaatcggtgatctttttttttttttttttttttttttttctttttggataaatctcaatgggtgatctattgactgtttctctacgtcactattttacttacgtcatagatgtggcggcatatattcttcaggacttttcagccataggcaagagtgatagagatactcatatgcatgaaacactaagaaacaaataattcttgactttttttcttttatttgaaaattgactgtagatataaacttttattttttctgactgtaaatataatcttaattgccaaactgtccgatacgatttttctgtattatttacaggaagatatcttcaaaacattttgaatgaagtaatatatgaaattcaaatttgaaatagaagacttaaattagaatcatgaagaaaaaaaaaacacaaaacaactgaatgacatgaaacaactatatacaatgtttcttaataaacttcatttagggtatacttacatatatactaaaaaaatatatcaacaatggcaaagctaccgatacgaaacaatattaggaaaaatgtgtgtaaggacaagattgacaaaaaaatagttacgaaaacaacttctattcatttggacaattgcaatgaatattactaaaatactcacacatggaccatgtatttacaaaaacgtgagatctatagttaacaaaaaaaaaaagaaaaaaatagttttcaaatctctatataagcgatgtttacgaaccccaaaatcataacacaacaataaccattatcaacttagaaaaatgactcaattcaatatcccagtaaccatgtcttcaagcttaagcataattttggtcattcttgtatctttgagcactgcacactcagagctatgcaacccacaagacaagcaagcccttctccaaatcaagaaagaccttggcaacccaaccactctctcctcatggcttccaaccaccgactgttgcaacagaacctggctaggtgttttatgcgacaccgacacccaaacatatcgcgtcaacaacctcgacctctccggccttaacctcccaaaaccctaccctatcccttcctccctcgccaacctcccctacctcaattttctatacattggtggcatcaataacctcgtcggtccaatcccccccgccatcgctaaactcacccaactccactatctctatatcacccacaccaatgtctccggcgcaatacccgatttcttgtcacagatcaaaaccctcgtcaccctcgacttctcctacaacgccctctccggcaccctacctccctccatctcttctctccccaacctcgtcggaatcacattcgacggcaaccgaatctccggcgccatccccgactcctacggctcattttcgaagctgttcacgtcgatgaccatctcccgcaaccgcctcaccgggaagattccgccgacgtttgcgaatctgaacctggcgttcgttgacttgtctcgaaacatgctggagggtgacgcgtcggtgttgttcggatcagataagaacacgcagaagatacatctggcgaagaactctcttgcctttgatttggggaaagtggggttgtcaaagaacttgaacgggttggatctgaggaacaaccgtatctatgggacgctaccgcagggactgacgcagctaaagtttctgcacagtttaaatgtgagcttcaacaatctgtgcggtgagattcctcaaggtgggaacttgcaaagatttgacgtttctgcttatgccaacaacaagtgcttgtgtggttctcctcttcctgcctgcactgcggccgcaccctgctccgtgactgagtactctggcctcgccaccgctgtctcatcctgcaaaaacatcgtgctcaacggtttccaagtcccgacaggcaagcaactcgacctatccagcctccagaatgactcgaccgttaccttcaagggcacgaccacttttgccaccactgctgataacgactttaatcctatcgtcattagtggaagtaacatcactatcactggtgcatctggccatgtcattgatggcaacggtcaggcgtactgggatggcaaaggttctaacagcaatagcaaccaaaagcccgatcacttcatcgttgttcagaagaccaccggcaactcaaagatcacaaacctaaatatccagaactggcccgttcactgcttcgacattacaggcagctcgcaattgaccatctcagggcttattcttgataacagagctggcgacaagcctaacgccaagagcggtagcttgcccgctgcgcataacaccgacggtttcgacatctcgtccagtgaccacgttacgctggataacaatcatgtttataaccaagatgattgtgttgctgttacttccggtacaaacatcgtcgtttctaacatgtattgctccggcggccatggtcttagtatcggatctgttggtggaaagagcgacaatgtcgttgatggtgttcagttcttgagctcgcaggttgtgaacagtcagaatggatgtcgcatcaagtccaactctggcgcaactggcacgatcaacaacgtcacctaccagaacattgctctcaccaacatcagcacgtacggtgtcgatgttcagcaggactatctcaacggcggccctactggaaagccgaccaacggagtcaagatcagcaacatcaagttcatcaaggtcactggcactgtggctagctctgcccaggattggtttattctgtgtggtgatggtagctgctctggatttaccttctctggaaacgctattactggtggtggcaagactagcagctgcaactatcctaccaacacttgccccagctag

SEQ ID NO: 10 - protein, 29 aa, Artificial Sequence
  synthetic primer
ATCGATGAATTCGAGCTATGCAACCCACA

SEQ ID NO: 11 - DNA, 35 bp, Artificial Sequence
  synthetic primer
tcttctaagtgcggccgcagtgcaggcaggaagag

SEQ ID NO: 12 - DNA, 37 bp, Artificial Sequence
  synthetic primer
tcaacactatgcggccgcaccctgctccgtgactgag

SEQ ID NO: 13 - DNA, 31 bp, Artificial Sequence
  synthetic primer
atcgattctagactagctggggcaagtgttg

SEQ ID NO: 14 - DNA, 44 bp, Artificial Sequence
  synthetic primer
actaagcctaggactatctagaatgactcaattcaatatcccag

SEQ ID NO: 15 - DNA, 16 bp, Artificial Sequence
  synthetic primer
ggggatggcgccggag

SEQ ID NO: 16 - DNA, 34 bp, Artificial Sequence
  synthetic primer
actaagctcgagatgactcaattcaatatcccag

SEQ ID NO: 17 - DNA, 33 bp, Artificial Sequence
  synthetic primer
cctaagttaattaactagctggggcaagtgttg

SEQ ID NO: 18 - DNA, 31 bp, Artificial Sequence
  synthetic primer
gactatctagaatgactcaattcaatatccc

SEQ ID NO: 19 - DNA, 22 bp, Artificial Sequence
  synthetic primer
gttagcacaagcttgttttaac

SEQ ID NO: 20 - DNA, 34 bp, Artificial Sequence
  synthetic primer
cctaagtctagaggtcttaattaactagctgggg

SEQ ID NO: 21 - DNA, 65 bp, Artificial Sequence
  synthetic primer
tgcttaagcttgaagacatggttactgggatattgaattgagtcatttttctaagttgataatgg

SEQ ID NO: 22 - protein, 373 aa, Fusarium phyllophilum
MVRNIVSRLCSQLFALPSSSLQERDPCSVTEYSGLATAVSSCKNIVLNGFQVPTGKQLDLSSLQNDSTVTFKGTTTFATTADNDFNPIVISGSNITITGASGHVIDGNGQAYWDGKGSNSNSNQKPDHFIVVQKTTGNSKITNLNIQNWPVHCFDITGSSQLTISGLILDNRAGDKPNAKSGSLPAAHNTDGFDISSSDHVTLDNNHVYNQDDCVAVTSGTNIVVSNMYCSGGHGLSIGSVGGKSDNVVDGVQFLSSQVVNSQNGCRIKSNSGATGTINNVTYQNIALTNISTYGVDVQQDYLNGGPTGKPTNGVKISNIKFIKVTGTVASSAQDWFILCGDGSCSGFTFSGNAITGGGKTSSCNYPTNTCPS

SEQ ID NO: 23 - protein, 342 aa, Phaseolus vulgaris
MTQFNIPVTMSSSLSIILVILVSLRTALSELCNPQDKQALLQIKKDLGNPTTLSSWLPTTDCCNRTWLGVLCDTDTQTYRVNNLDLSGHNLPKPYPIPSSLANLPYLNFLYIGGINNLVGPIPPAIAKLTQLHYLYITHTNVSGAIPDFLSQIKTLVTLDFSYNALSGTLPPSISSLPNLGGITFDGNRISGAIPDSYGSFSKLFTAMTISRNRLTGKIPPTFANLNLAFVDLSRNMLEGDASVLFGSDKNTKKIHLAKNSLAFDLGKVGLSKNLNGLDLRNNRIYGTLPQGLTQLKFLQSLNVSFNNLCGEIPQGGNLKRFDVSSYANNKCLCGSPLPSCT

SEQ ID NO: 24 - protein, 362 aa, Aspergillus niger
MHSFASLLAYGLVAGATFASASPIEARDSCTFTTAAAAKAGKAKCSTITLNNIEVPAGTTLDLTGLTSGTKVIFEGTTTFQYEEWAGPLISMSGEHITVTGASGHLINCDGARWWDGKGTSGKKKPKFFYAHGLDSSSITGLNIKNTPLMAFSVQANDITFTDVTINNADGDTQGGHNTDAFDVGNSVGVNIIKPWVHNQDDCLAVNSGENIWFTGGTCIGGHGLSIGSVGDRSNNVVKNVTIEHSTVSNSENAVRIKTISGATGSVSEITYSNIVMSGISDYGVVIQQDYEDGKPTGKPTNGVTIQDVKLESVTGSVDSGATEIYLLCGSGSCSDWTWDDVKVTGGKKSTACKNFPSVASC

SEQ ID NO: 25 - protein, 342 aa, Phaseolus vulgaris
MTQFNIPVTMSSSLSIILVILVSLRTALSELCNPQDKQALLQIKKDLGNPTTLSSWLPTTDCCNRTWLGVLCDTDTQTYRVNNLDLSGHNLPKPYPIPSSLANLPYLNFLYIGGINNLVGPIPPAIAKLTQLHYLYITHTNVSGAIPDFLSQIKTLVTLDFSYNALSGTLPPSISSLPNLVGITFDGNRISGAIPDSYGSFSKLFTSMTISRNRLTGKIPPTFANLNLAFVDLSRNMLQGDASVLFGSDKNTQKIHLAKNSLDFDLEKVGLSKNLNGLDLRNNRIYGTLPQGLTQLKFLHSLNVSFNNLCGEIPQGGNLQRFDVSAYANNKCLCGSPLPACT

SEQ ID NO: 26 - protein, 330 aa, Malus domestica
MELKFSIFLSLTLLFSSVLKPALSDLCNPDDKKVLLQIKKAFGDPYVLTSWKSDTDCCDWYCVTCDSTTNRINSLTIFAGQVSGQIPALVGDLPYLETLEFHKQPNLTGPIQPAIAKLKGLKFLRLSWTNLSGSVPDFLSQLKNLTFLDLSFNNLTGAIPSSLSQLPNLNALHLDRNKLTGHIPKSLGQFIGNVPDLYLSHNQLSGNIPTSFAQMDFTSIDLSRNKLEGDASVIFGLNKTTQIVDLSRNLLEFNLSKVEFPTSLTSLDINHNKIYGSIPVEFTQLNFQFLNVSYNRLCGQIPVGGKLQSFDEYSYFHNRCLCGAPLPSCK

SEQ ID NO: 27 - protein, 362 aa, Colletotrichum lupini var setosum
MVSSLLALGALAATAIAAPLDARASCTFTDAAAAIKGKASCTSIILNGIVVPAGTTLDMTGLKSGTTVTFQGKTTFGYKEWEGPLISFSGTNININGASGHSIDCQGSRWWDSKGSNGGKTKPKFFYAHSLKSSNIKGLNVLNTPVQAFSINSATTLGVYDVIIDNSAGDSAGGHNTDAFDVGSSTGVYISGANVKNQDDCLAINSGTNITFTGGTCSGGHGLSIGSVGGRSDNTVKTVTISNSKIVNSDNGVRIKTVSGATGSVSGVTYSGITLSNIAKYGIVIEQDYENGSPTGTPTNGVPITGLTLSKITGSVASSGTNVYILCASGACSNWKWSGVSVTGGKKSTKCSNIPSGSGAAC

SEQ ID NO: 28 - protein, 333 aa, Vitis vinifera
METSKLFLLSSSLLLVLLATRPCPSLSERCNPKDKKVLLQIKKALDTPYILASWNPNTDCCGWYCVECDLTTHRINSLTIFSGQLSGQIPDAVGDLPFLETLIFRKLSNLTGQIPPAIAKLKHLKMVRLSWTNLFGPVPAFFSELKNLTYLDLSFNNLSGPIPGSLSLLPNLGALHIDRNHLTGPIPDSFGKFAGSTPGLHLSHNQLSGKIPYSFRGFDPNVMDLSRNKLEGDLSIFFNANKSTQIVDFSRNLFQFDLSRVEFPKSLTSLDLSHNKIAGSLPEMMTSLDLQFLNVSYNRLCGKIPVGGKLQSFDYDSYFHNRCLCGAPLQSCK

SEQ ID NO: 29 - protein, 374 aa, Botrytis cinerea
MVHITSLISFLASTALVSAAPGSAPADLDRRAGCTFSTAATAIASKTTCSTIILDSVVVPAGTTLDLTGLKTGTKVIFQGTATFGYSEWEGPLISISGQDIVVTGASGNKIDGGGARWWDGLGSNVSAGKGKVKPKFFSAHKLTGSSSITGLNFLNAPVQCISIGQSVGLSLININIDNSAGDAGNLGHNTDAFDINLSQNIFISGAIVKNQDDCVAVNSGTNITFTGGNCSGGHGLSIGSVGGRSGTGANDVKDVRFLSSTVQKSTNGVRVKTVSDTKGSVTGVTFQDITLIGITGVGIDVQQDYQNGSPTGTPTNGVPITGLTMNNVHGNVIGGQNTYILCANCSGWTWNKVAVTGGTVKKACAGVPTGASC

SEQ ID NO: 30 - protein, 330 aa, Arabidopsis thaliana
MDKTATLCLLFLFTFLTTCLSKDLCNQNDKNTLLKIKKSLNNPYHLASWDPQTDCCSWYCLECGDATVNHRVTALTIFSGQISGQIPAEVGDLPYLETLVFRKLSNLTGTIQPTIAKLKNLRMLRLSWTNLTGPIPDFISQLKNLEFLELSFNDLSGSIPSSLSTLPKILALELSRNKLTGSIPESFGSFPGTVPDLRLSHNQLSGPIPKSLGNIDFNRIDLSRNKLQGDASMLFGSNKTTWSIDLSRNMFQFDISKVDIPKTLGILDLNHNGITGNIPVQWTEAPLQFFNVSYNKLCGHIPTGGKLQTFDSYSYFHNKCLCGAPLEICK

SEQ ID NO: 31 - protein, 330 aa, Arabidopsis thaliana
MDKTMTLFLLLSTLLLTTSLAKDLCHKDDKTTLLKIKKSLNNPYHLASWDPKTDCCSWYCLECGDATVNHRVTSLIIQDGEISGQIPPEVGDLPYLTSLIFRKLTNLTGHIQPTIAKLKNLTFLRLSWTNLTGPVPEFLSQLKNLEYIDLSFNDLSGSIPSSLSSLRKLEYLELSRNKLTGPIPESFGTFSGKVPSLFLSHNQLSGTIPKSLGNPDFYRIDLSRNKLQGDASILFGAKKTTWIVDISRNMFQFDLSKVKLAKTLNNLDMNHNGITGSIPAEWSKAYFQLLNVSYNRLCGRIPKGEYIQRFDSYSFFHNKCLCGAPLPSCK